(19) United States
(12) Patent Application Publication
(10) Pub. No.: US 2025/0302990 A1
(43) Pub. Date: Oct. 2, 2025

(54) ENHANCING NON-VIRAL DNA DELIVERY AND EXPRESSION

(71) Applicant: Spark Therapeutics, Inc., Philadelphia, PA (US)

(72) Inventors: Pedro Cejas, Oreland, PA (US); Matthew Walsh, Philadelphia, PA (US); Sean Armour, Holland, PA (US); Rui Zhang, Winchester, MA (US)

(21) Appl. No.: 19/109,831

(22) PCT Filed: Sep. 7, 2023
(86) PCT No.: PCT/US2023/073601

§ 371 Date: Mar. 7, 2025

Related U.S. Application Data
(60) Provisional application No. 63/375,156, filed on Sep. 9, 2022.

(51) Int. Cl. A61K 48/00; A61K 9/51; A61K 31/495; A61K 39/00; C07D 241/08; C07K 16/24

(52) U.S. Cl. CPC A61K 48/0033; A61K 9/5123; A61K 31/495; C07D 241/08; C07K 16/249; A61K 2039/505; C07K 2317/76

(57) ABSTRACT

The present invention features method and composition that can be used to facilitate intracellular delivery of DNA to a subject. The provided methods and compositions employ a nanoparticle for intracellular DNA delivery and a type 1 interferon receptor pathway inhibitor. The type 1 interferon receptor pathway inhibitor is provided to decrease the subject's immune response that can be stimulated by the DNA.

### CROSS REFERENCE TO RELATED APPLICATION

The present application claims priority to U.S. Provisional Application No. 63/375,156 filed on Sep. 9, 2022, the disclosure of which is incorporated herein by reference in its entirety.

### REFERENCE TO SEQUENCE LISTING SUBMITTED ELECTRONICALLY

The contents of the electronic sequence listing (065830_17WO1.xml; Size: 3,359 bytes; and Date of Creation: Sep. 6, 2023) is herein incorporated by reference in its entirety.

### BACKGROUND OF THE INVENTION

Gene therapy involves using nucleic acid to modify a subject's DNA to achieve a beneficial effect. Gene modification can be performed using different strategies including gene augmentation, gene suppression and genome editing. (Anguela and High Annu. Rev. Med. 2019, 70, 73; and Li et al., Signal Transduction and Targeted Therapy 2020, 5, 1.)

An effective delivery system for nucleic acid is important for successful gene therapy. Successful delivery of the nucleic acid provides a sufficient amount to a target cell to achieve a beneficial effect without producing an unacceptable adverse reaction. The delivery system should protect the genetic material from enzymatic degradation, have a sufficiently long lifetime in the body, be able to reach the site within the body where it is needed, have tolerable toxicity, and be able to cross the cell membrane.

Gene therapy vectors can be broadly categorized as viral and non-viral. Each type of vector has advantages and disadvantages. Viral vectors are generally more efficient at delivering genetic material to a cell, but have a greater potential for immunogenicity, toxin production and insertional mutagenesis, and a more limited transgenic capacity size. Advantages of non-viral vectors include greater transgene capacity, the ability to dose subjects with pre-existing antibodies to vector capsid, and greater ability to re-dose a subject. Challenges associated with non-viral delivery can include lower transfection efficiency, potential nucleic acid degradation, innate immunity, low efficiency of gene delivery to somatic targets and lower in vivo gene expression levels than viral approaches. (Hardee et al., Genes (2017) 8, 65 and Nayerossadat et al., Adv. Biomed Res. (2012) 1, 27.)

### BRIEF SUMMARY OF THE INVENTION

The present invention features methods and compositions that can be used in methods involving intracellular delivery of DNA to a subject. The provided methods and compositions employ a nanoparticle for intracellular DNA delivery, and a type 1 interferon receptor pathway inhibitor. The type 1 interferon receptor pathway inhibitor is provided to decrease the subject's immune response that can be stimulated by the DNA.

Thus, a first aspect of the present invention describes a method of intracellular delivery of DNA comprising administering to a subject:
a) a type 1 interferon receptor pathway inhibitor; and
b) a first nanoparticle comprising the DNA, wherein step (b) can be performed prior to, concomitantly with, or after step (a).

Another aspect of the present invention describes a nanoparticle comprising (a) a DNA and (b) a type 1 interferon receptor pathway inhibitor.

Additional aspects of the present invention include pharmaceutical compositions containing the nanoparticles, inhibitors and DNA vectors described herein, pharmaceutical compositions for uses described herein, and preparation of medicaments for uses described herein. Pharmaceutical compositions for uses described herein can provide a DNA vector comprising a transgene for use in a patient that is administered prior to, concomitantly with, of after, a type 1 interferon receptor pathway inhibitor. Similarly, preparation of medicaments for uses described herein can involve preparation of a pharmaceutical composition containing a DNA vector comprising a transgene for use in a patient that prior to, concomitantly with, or after, is administered a type 1 interferon receptor pathway inhibitor; or preparation of a pharmaceutical composition containing a type 1 interferon receptor pathway inhibitor DNA for use in a patient that prior to, concomitantly with, or after, is administered a DNA vector comprising a transgene.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates survival of wild-type (WT) mice and interferon receptor alpha receptor (IFNAR)-deficient (IFNAR KO) mice dosed systemically at t=0, week 7, and week 12 with 1.25 mpk (25 μg) of human factor IX (hFIX) transgene-encoding plasmid DNA encapsulated in lipid nanoparticles.

FIG. 2 illustrates hFIX levels of WT mice and IFNAR-deficient (IFNAR KO) mice dosed systemically at t-0, week 7, and week 12 with 1.25 mpk (25 μg) of human factor IX (hFIX) transgene-encoding plasmid DNA encapsulated in lipid nanoparticles.

FIGS. 3A-3C illustrate cytokine levels in WT mice dosed systemically at week 6 (day 41) post-primary dosing with 1.25 mpk (25 μg) of human factor IX (hFIX) transgene-encoding plasmid DNA encapsulated in lipid nanoparticles and either untreated or treated once with 15 mpk anti-mouse IFNAR blocking antibody (“+aIFNAR”). Plasma cytokine levels were assayed at 4 hours post day 41 dosing and compared to pooled plasma pre-dosing levels (“Baseline”). FIG. 3A illustrates IL-6 levels, FIG. 3B illustrates IFN alpha levels and FIG. 3C illustrates IFN gamma levels. “LLOQ” refers to lower limit of quantification and “ULOQ” refers to upper limit of quantification.

FIG. 4 illustrates survival of WT mice dosed systemically at t-0 with 1.25 mpk (25 μg) and at day 41 with 2.5 mpk (50 μg) of human factor IX (hFIX) transgene-encoding plasmid DNA encapsulated in lipid nanoparticles; and either untreated or treated once before each dosing with 15 mpk anti-mouse IFNAR blocking antibody.

FIG. 5 illustrates hFIX levels in WT mice dosed systemically at t-0 with 1.25 mpk (25 μg) and at day 41 with 2.5 mpk (50 μg) of human factor IX (hFIX) transgene-encoding plasmid DNA encapsulated in lipid nanoparticles; and either untreated or treated once with 15 mpk anti-mouse IFNAR blocking antibody.

FIG. 6 illustrates survival of WT mice dosed systemically with 2.5 mpk (50 μg) of human factor IX (hFIX) transgene-encoding plasmid DNA encapsulated in lipid nanoparticles; and either untreated or treated with ruxolitinib or baricitinib orally 30 minutes prior to, and on days 1, 2, and 3 post systemic dosing with hFIX transgene encoding plasmid DNA encapsulated in lipid nanoparticles.

FIG. 7 illustrates FIX protein levels in blood plasma of mice not treated; or treated orally with baricitinib (“Bar”) 90 minutes prior to DNA-LNP dosing, and then daily on days 1-6 post-DNA-LNP dosing. Transgenic hFIX protein in blood plasma of surviving mice was measured by ELISA one-week post-DNA-LNP dosing.

FIG. 8 illustrates FIX protein levels in Wild type C57BL/6 (“WT”) mice and STING-deficient mice (i.e., mice harboring the Goldenticket nonsense mutation (“STING (Gt)”)). Mice were untreated, or treated IP once with 15 mpk anti-mouse IFNAR blocking antibody (“anti-IFNAR”) 3 hours prior to systemic (IV tail injections) dosing with 5 mpk (100 μg) of human factor IX (hFIX) transgene-encoding plasmid DNA encapsulated in lipid nanoparticles (DNA-LNP). Transgenic hFIX protein in blood plasma of surviving mice was measured by ELISA four weeks post-DNA-LNP dosing. “LLOQ” refers to lower limit of quantification and “ULOQ” refers to upper limit of quantification.

FIG. 9A and FIG. 9B illustrate the effect of an anti-IFNAR antibody delivered by different LNPs on transgene FIX expression in C57BL/6 mice. FIG. 9A illustrates results using Gen Voy-ILM™. FIG. 9B illustrates results using LPN comprising Compound 9. “LLOQ” refers to lower limit of quantification.

FIG. 10 illustrates the effect of an anti-IFNAR antibody delivered by an LNP on transgene erythropoietin (EPO) expression in Balb/c mice. “LLOQ” refers to lower limit of quantification and “ULOQ” refers to upper limit of quantification.

### DETAILED DESCRIPTION OF THE INVENTION

The present invention features methods and compositions for intracellular DNA delivery to a subject employing a nanoparticle comprising the DNA, and a type 1 interferon receptor pathway inhibitor. Intracellular DNA delivery has different uses including delivery of a DNA vector into a subject for transgene expression. As illustrated in the examples below potential benefits of inhibiting the type 1 interferon receptor pathway inhibitor include enhanced transgene expression and reduced cytokine production, including IFN gamma production.

A “type 1 interferon receptor pathway inhibitor” refers to a compound inhibiting Type 1 interferon receptor (IFNAR) activity and/or signal transduction from the type 1 interferon receptor. Type 1 interferon receptor pathway inhibitors include compounds inhibiting IFNAR receptor activity, and downstream activities such as those mediated by Janus activated kinase 1, a tyrosine kinase 2, or by a signal transducer and activator of transcription (STAT) protein.

A “nanoparticle” refers to a small non-viral particle that can encapsulate or associate with DNA and facilitates DNA delivery to a cell. The nanoparticle may also be used to deliver, for example, different DNA vectors, different transgenes, type 1 interferon receptor pathway inhibitors, cytosolic DNA-sensing inhibitors, and immune cell modulators. The nanoparticle ranges in size from about 10 nm to about 1000 nm. In different embodiments, the nanoparticle is about 50 nm to about 500 nm, or about 50 nm to about 200 nm.

Reference to “subject” indicates a mammal, including humans; non-human primates such as apes, gibbons, gorillas, chimpanzees, orangutans, macaques; domestic animals, such as dogs and cats; farm animals such as poultry and ducks, horses, cows, goats, sheep, and pigs; and experimental animals such as mice, rats, rabbits, guinea pigs. A preferred subject is a human subject being treated. However, a subject can also include animal disease models, for example, mouse and other animal models of protein/enzyme deficiencies such as Pompe disease (loss of GAA), and glycogen storage diseases (GSDs).

References to “DNA vector” indicates a DNA polymer containing a transgene operative linked to one more regulatory element providing for RNA expression from the transgene. The produced RNA can itself be functional or can encode for a protein. One type of regulatory element is a promoter, which binds RNA polymerase and the necessary transcription factors to initiate transcription. When encoding for protein, the produced RNA sequence will also encode a termination sequence at the end of the coding sequence. Additional regulatory elements include those impacting RNA expression, RNA stability, and protein production. DNA vectors may be single-stranded, double-stranded, or contain a combination of single and double-stranded regions. The DNA vector may also include more than one transgene and multiple regulatory elements of the same or different types.

DNA refers to a DNA polymer and includes double-stranded DNA, single-stranded DNA, and DNA having single and double-stranded regions.

Reference to the DNA, which includes DNA making up a vector, “substantially” comprise, comprises, or comprising “double-stranded DNA” indicates more than half, at least 75%, at least 90%, at least 95%, or at least 99% of the DNA is double-stranded DNA or 100% of the DNA is double-stranded. Similarly, reference to the DNA, which includes DNA making up a vector, “substantially” comprise, comprises, or comprising “single-stranded DNA” indicates more than half, at least 75%, at least 90%, at least 95%, or at least 99% of the DNA is single-stranded DNA or 100% of the DNA is single-stranded.

The term “operatively linked” refers to the association of two or more nucleic acid segments on a single DNA where the function of one is affected by the other.

Reference to “transgene” indicates a DNA region capable of being expressed to RNA, without regard to origin of the transgene sequence. The transgene is generally part of a longer length DNA, where the DNA contains at least one region with which the transgene is not normally associated with in nature.

The singular forms “a”, “an”, and “the” include plural reference unless the context clearly dictates otherwise.

As used herein, the conjunctive term “and/or” between multiple recited elements is understood to encompass both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,”, “such as” followed by different members or examples, are open-ended descriptions where the listed members or examples are illustrative and other member or examples can be provided or used.

The terms “polypeptides,” “proteins” and “peptides” can be used interchangeably to refer to an amino acid sequence without regard to function. Polypeptides and peptides contain at least two amino acids, while proteins contain at least about 10 amino acid acids. The provided amino acids include naturally occurring amino acids and amino acids provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein reference to an open-ended term such as “comprising” can be replaced by the terms “consisting” or “consisting essentially of.”

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100%, as well as 95.1%, 95.2%, 95.3%, 95.4%, 95.5%, etc., 96.1%, 96.2%, 96.3%, 96.4%, 96.5% and so forth; reference to a numerical range, such as “1-4” includes 1, 2, 3, as well as 1.1, 1.2, 1.3, 1.4 and so forth; reference to “1 to 4 weeks” includes 7, 8, 9, 10, 11, 12, 13, 14, 15, 16, 17, 18, 19, 20, 21, 22, 23, 24, 25, 26, 27, or 28 days; reference to a numerical range, such as “0.01 to 10” includes 0.011, 0.012, 0.013 and so forth, as well as 9.5, 9.6, 9.7, 9.8, 9.9 and 10 and so forth. For example, a dosage of “0.01 mg/kg to 10 mg/kg” body weight of a subject includes 0.011 mg/kg, 0.012 mg/kg, 0.013 mg/kg, 0.014 mg/kg, 0.015 mg/kg and so forth as well as 9.5 mg/kg, 9.6 mg/kg, 9.7 mg/kg, 9.8 mg/kg, 9.9 mg/kg and so forth.

Reference to an integer with more (greater) or less than includes numbers greater or less than the reference number, respectively. Thus, for example, reference to more than 2 includes 3, 4, 5, 6, 7, 8, 9, 10, 11, 12, 13, 14, 15; and administration “two or more” times includes 2, 3, 4, 5, 6, 7, 8, 9, 10, 11, 12, 13, 14, 15, or more times.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides various embodiments. These separations should not be considered as disconnecting the substance of a paragraph or section or embodiments from the substance of another paragraph or section or embodiment. The provided descriptions have broad application and encompass all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

While certain combination of features is highlighted herein, all of the features disclosed herein can be combined in any combination.

### I. Nanoparticles

A variety of different nanoparticles can be employed including lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. (See, e.g., Riley and Vermerris Nanomaterials 2017, 7, 94; Thomas et al., Molecules 2019, 24, 3744; Bochicchio et al., Pharmaceutics 2021, 13, 198; Munagala et al., Cancer Letters 2021, 505, 58; Fu et al., 2020 NanoImpact 20, 100261; and Neshat et al. Current Opin. Biotechnol. 2020, 66:1-10.)

If desired, a nanoparticle can be targeted to a cell type using, for example, targeting ligands recognizing a target cell receptor. Examples of targeting ligands include carbohydrates (e.g., galactose, mannose, glucose, and galactomannan), endogenous ligands (e.g., folic acid and transferrin), antibodies (e.g., anti-HER2 antibody and hD1) and protein/peptides (e.g., RGD, epidermal growth factor, and low-density lipoprotein) and peptides. (See, e.g., Teo et al., Advanced Drug Delivery Reviews 2016, 98, 41.)

The present application features the use of nanoparticles to deliver DNA. In different embodiments, nanoparticles can deliver additional compounds such as type 1 interferon receptor pathway inhibitors, cytosolic DNA-sensing inhibitors, immunosuppressants, phagocyte depleting compounds, and additional therapeutic compounds; one or more additional compounds is provided in different nanoparticles; and one or more additional compounds is provided in the same nanoparticle as the DNA vector, for example a DNA vector and a type 1 interferon receptor pathway inhibitor; or a DNA vector, a type 1 interferon receptor pathway inhibitor, a cytosolic DNA-sensing inhibitor and an immune cell modulator. Reference to “compounds” includes small molecules and large molecules (e.g., therapeutic proteins and antibodies), and nucleic acid.

The production of different nanoparticles and incorporation of nucleic acid and other compounds is well known in the art, and exemplified by different publications throughout the discussion in Section I. In general, exposure kinetics of nanoparticle cargoes (e.g., the DNA and/or inhibitor) can be affected by providing DNA and different compounds with different environments or association with different structures.

Examples of publications illustrating incorporation of nucleic acid in a particular nanoparticle such as an LPNP and a LNP include Teo et al., Advanced Drug Delivery Reviews 2016, 98, 41; Bochicchio et al., Pharmaceutics 2021, 13, 198; Mahzabin and Das, IJPSR 2021, 12(1), 65; and Teixeira et al., Progress in Lipid Research 2017, 1 (each of which are hereby incorporated by reference herein in their entirety). Such references also point out an advantage of LPNP in providing different structures interacting with nucleic acid and small molecules that can impact desired release kinetics. Factors that may impact small molecule incorporation into a nanoparticle include hydrophobicity and the presence of an ionizable moiety. (See, e.g., Nii and Ishii, International Journal of Pharmaceutics 2005, 298, 198; and Chen et al., Journal of Controlled Release 2018, 286, 46.)

In an embodiment, a compound (e.g., type 1 interferon receptor pathway inhibitor, cytosolic DNA-sensing inhibitor and/or immune cell modulator) is linked to a fatty acid to increase hydrophobicity. Examples of fatty acids that can linked to small molecules include those described by Chen et al., Journal of Controlled Release 2018, 286, 46-54.

### I.A. Lipid-Based Delivery Systems

Lipid-based delivery systems include the use of a lipid as a component. Examples of lipid-based delivery systems include liposomes, LNPs, micelles, and extracellular vesicles.

A “lipid nanoparticle” or “LNP” refers to a lipid-based vesicle useful for delivery of nucleic acid molecules and having dimensions on the nanoscale. In different embodiments the nanoparticle is from about 10 nm to about 1000 nm, about 50 nm to about 500 nm, or about 50 nm to about 200 nm.

DNA is negatively charged. Thus, it can be beneficial for the LNP to comprise a cationic lipid such as, for example, an amino lipid. Exemplary amino lipids are described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids described in U.S. Pat. No. 9,512,073, hereby incorporated herein in its entirety.

The terms “cationic lipid” and “amino lipid” are used interchangeably herein to include lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipid can also be titratable cationic lipids. In certain embodiments, the cationic lipids comprise a protonatable tertiary amine (e.g., pH-titratable) group; C18 alkyl chains, wherein each alkyl chain independently can have one or more double bonds, one or more triple bonds; and ether, ester, or ketal linkages between the head group and alkyl chains.

Cationic lipids include 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino) butanoate (DLin-M-C3-DMA, also known as MC3), salts thereof, and mixtures thereof. Other cationic lipids also include 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids include 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DODAC), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N-(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido) ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Additional ionizable lipids that can be used include C12-200, 3060i10, MC3, cKK-E12, ATX-002, ATX-003, and Merck-32. U.S. Patent Application Publication No. 2017/0367988, describes Merck-32.

In further embodiments, cationic lipids can be present in an amount from about 10% by molar ratio of the LNP to about 85% by molar ratio of the LNP, or from about 50% by molar ratio of the LNP to about 75% by molar ratio of the LNP.

LNPs can comprise a neutral lipid. Neutral lipids can comprise a lipid species existing either in an uncharged or neutral zwitterionic form at physiological pH. Such lipids include diacylphosphatidylcholine, diacylphosphatidylethanolamine, ceramide, sphingomyelin, dihydrosphingomyelin, cephalin, and cerebrosides. The selection of neutral lipids is generally guided by considerations including particle size and stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized. In certain embodiments, lipids containing saturated fatty acids with carbon chain lengths in the range of C14 to C22 can be used. In certain embodiments lipids with mono or di-unsaturated fatty acids with carbon chain lengths in the range of C14 to C22 are used. Additionally, lipids having mixtures of saturated and unsaturated fatty acid chains can be used. Exemplary neutral lipids include 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or a phosphatidylcholine. The neutral lipids can also be composed of sphingomyelin, dihydrosphingomyelin, or phospholipids with other head groups, such as serine and inositol.

In further embodiments, providing for neutral lipids, the neutral lipid can be present in an amount from about 0.1% by weight of the LNP to about 99% by weight of the LNP, or from about 5% by weight of the LNP to about 15% by weight of the LNP, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNPs can contain additional components such as sterols and polyethylene glycol. Sterols can confer fluidity to the LNP. As used herein “sterol” refers to a naturally occurring sterol of plant (phytosterols) or animal (zoosterols) origin as well as non-naturally occurring synthetic sterols, all of which are characterized by the presence of a hydroxyl group at the 3-position of the steroid A-ring. Suitable sterols include those conventionally used in the field of liposome, lipid vesicle or lipid particle preparation, most commonly cholesterol. Phytosterols include campesterol, sitosterol, and stigmasterol. Sterols also include sterol-modified lipids, such as those described in U.S. Patent Application Publication 2011/0177156. In different embodiments providing for a sterol, the sterol is present in an amount from about 1% by weight of the LNP to about 80% by weight of the LNP or from about 10% by weight of the LNP to about 25% by weight of the LNP.

Polyethylene glycol (PEG) is a water-soluble polymer of ethylene PEG repeating units, and can be linear or branched. PEGs are classified by their molecular weights, for example, PEG 2000 has an average molecular weight of about 2,000 Daltons, and PEG 5000 has an average molecular weight of about 5,000 Daltons. PEGs commercially available from Sigma Chemical Co. and other companies include monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments concerning PEG, PEG has an average molecular weight of about 550 to about 10,000 Daltons and is optionally substituted by alkyl, alkoxy, acyl or aryl. In further embodiments, the PEG is substituted with methyl at the terminal hydroxyl position. In further embodiments, the PEG has an average molecular weight from about 750 to about 5,000 Daltons, or from about 1,000 to about 5,000 Daltons, or from about 1,500 to about 3,000 Daltons, or from about 2,000 Daltons, or from about 750 Daltons.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.1% by weight of the LNP to about 50% by weight of the LNP, or from about 5% by weight of the LNP to about 15% by weight of the LNP.

In further embodiments concerning LNP size, prior to encapsulating nucleic acid, LNPs have a size range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or Billingsley et al., International Patent Publication No. WO 2021/077066 (both of which are hereby incorporated by reference herein in their entirety). Billingsley et al., and WO2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

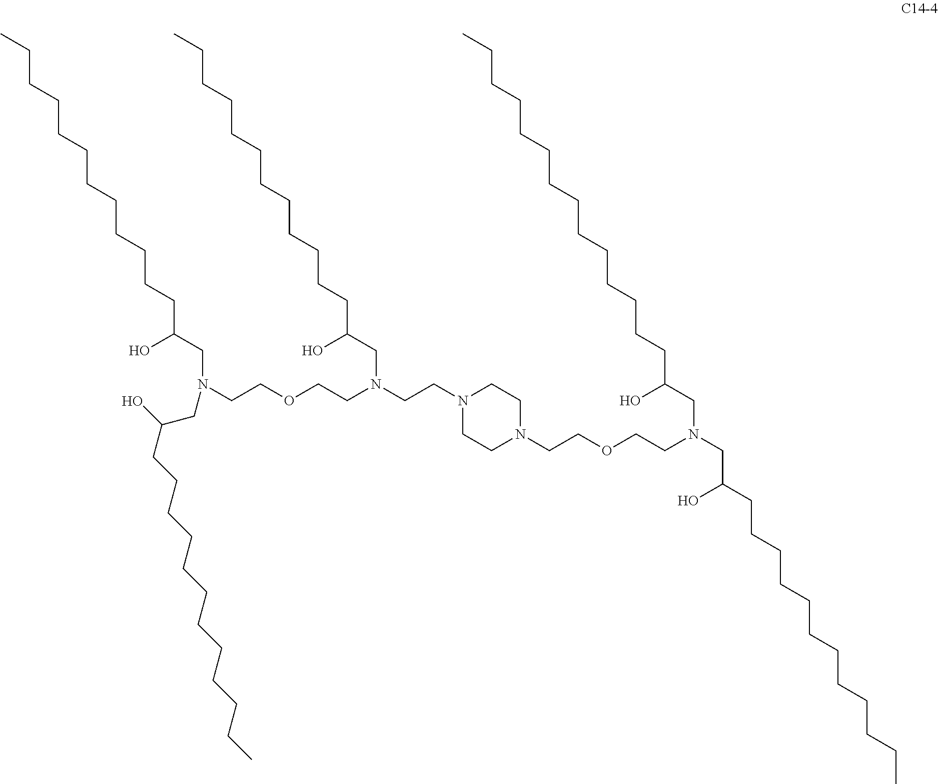 [SMILES: CCCCCCCCCCCCCCC(O)CN(CCOCCN1CCN(CCN(CCOCCN(CC(O)CCCCCCCCCCCC)CC(O)CCCCCCCCCCCC)CC(O)CCCCCCCCCCCC)CC1)CC(O)CCCCCCCCCCCC]

In certain embodiments the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipids. In certain embodiments the LNP contains cKK-E12 (Dong et al., PNAS (2014) 111(11), 3955):

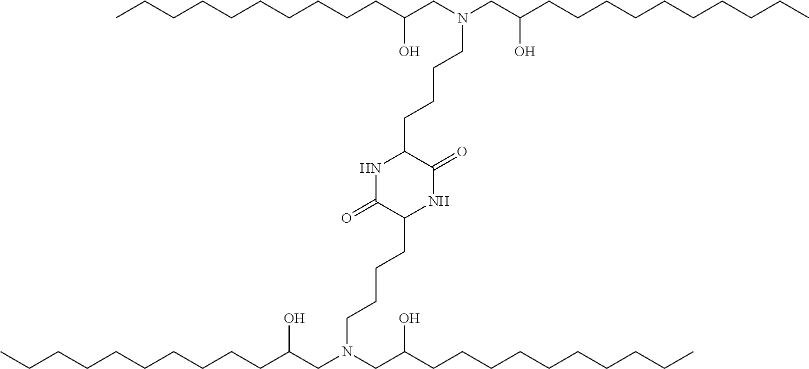 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In certain embodiments the LNP comprises a modified form of cKK-E12 referred to herein as “bCKK-E12,” having the following structure:

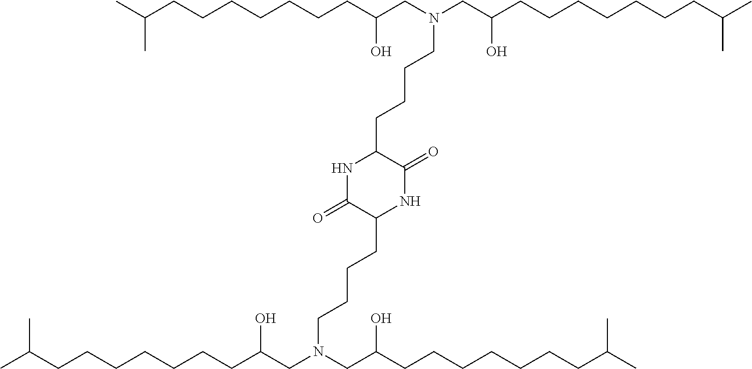 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

Certain embodiments are directed to bCKK-E12 or a pharmaceutically acceptable salt thereof. In certain embodiments the salt is an acid addition salt such as hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In certain embodiments the LNP comprises Lipid 1, 2, 3, 4, 5, 6, 7, 8, 9, or 10 as described by Sabnis et al., Molecular Therapy 2018, 26:6, 1509-1519 (hereby incorporated by reference herein in its entirety). In certain embodiments the LNP comprises Lipid 5, 8, 9, 10, or 11 described in Sabnis et al.

Lipid 5 of Sabnis et al. has the structure:

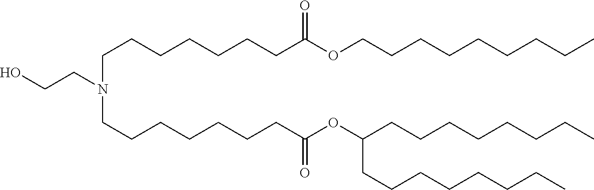 [SMILES: CCCCCCCCCOC(=O)CCCCCCCN(CCO)CCCCCCCC(=O)OC(CCCCCCCC)CCCCCCCC]

Lipid 9 of Sabnis et al. has the structure:

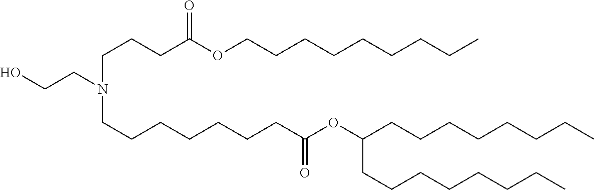 [SMILES: CCCCCCCCCOC(=O)CCCN(CCO)CCCCCCCC(=O)OC(CCCCCCCC)CCCCCCCC]

Additional lipids include those described in U.S. Patent Publication No. US2022204439 (hereby incorporated by reference herein in its entirety). One of the lipids described in U.S. Patent Publication No. US2022204439 is Compound 9:

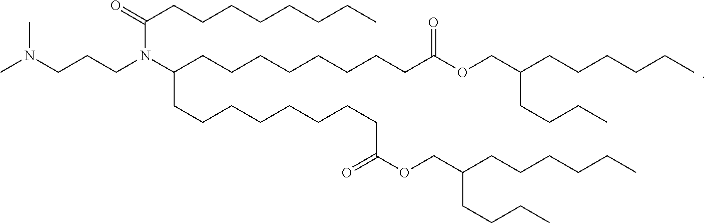 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

Additional lipids that may be utilized include those described by Roces et al., Pharmaceutics, 2020, 12,1095; Jayaraman et al, Angew. Chem. Int. Ed., 2012, 51, 8529-8533; Maier et al., www.moleculartherapy.org, 2013, Vol. 21, No. 8, 1570-1578; Liu et al., Adv. Mater. 2019, 31, 1902575, e.g., BAMEA-016B; Cheng et al., Adv. Mater., 2018, 30, 1805308, e.g., 5A2-SC8; Hajj and Ball, Small, 2019 15, 1805097, e.g., 3060i10; Du et al., U.S. Patent Application Publication No. 2016/0376224; and Tanaka et al., Adv. Funct. Mater., 2020, 30, 1910575; each of which are hereby incorporated by reference herein in their entirety.

### I.B. Additional Examples of Lipid-Based Delivery Systems

In further embodiments, LNP in mol % comprises, consists essentially, or consists, of the following components: (1) one or more cationic lipids from about 20% to about 65%, one or more phospholipid lipids from about 1% to about 50%, one or more PEG-conjugated lipid from about 0.1% to about 10%, and cholesterol from about 0% to about 70%; and (2) one or more cationic lipids from about 20% to about 50%, one or more phospholipid lipids from about 5% to about 20%, one or more PEG-conjugated lipids from about 0.1% to about 5%, and cholesterol from about 20% to about 60%. In further embodiments the phospholipid lipid is a neutral lipid; and the phospholipid lipid is DOPE or DSPC.

In further embodiments the LNP, in mole %, comprises, consists essentially, or consists of the following components: (1) cKK-E12 (further described in Section I.A. supra. about 35%; C14-PEG2000, about 2.5%; cholesterol, about 46.5%; and DOPE, about 16%; (2) bCKK-E12 (further described in Section I.A. supra.), about 35%; C14-PEG2000, about 2.5%; cholesterol, about 46.5%; and DOPE, about 16%; (3) Lipid 9 (further described in Sabnis et al. and Section I.A. supra.), about 50%; C14-PEG2000, about 1.5%; cholesterol, about 38.5%; and DSPC about 10%; (4) Lipid 5 (further described in Sabnis et al. and Section I.A. supra.), about 50%; C14-PEG2000 about 1.5%; cholesterol about 38.5%; and DSPC about 10%; and (5) ionizable lipid, about 50%; DSPC, about 10%; cholesterol, about 37.5%; and stabilizer (PEG-Lipid), about 2.5%; (6) is GenVoy-ILM™ LNP (Precision NanoSystems); or (7) Compound 9 (U.S. Patent Publication No. US2022204439) about 50%; C14-PEG2000, about 2.5%; cholesterol, about 37.5%; and DSPC, about 10%.

### I.C. Polymer-Based Nanoparticles

Polymer-based delivery systems can be made from a variety of different natural and synthetic materials. DNA and other compounds can be entrapped into the polymeric matrix of polymeric nanoparticles or can be adsorbed or conjugated on the surface of the nanoparticles. Examples of commonly used polymers for nucleic acid delivery include poly (lactic-co-glycolic acid) (PLGA), poly lactic acid (PLA), poly (ethylene imine) (PEI) and PEI derivatives, chitosan, dendrimers, polyanhydride, polycaprolactone, polymethacrylates, poly-L-lysine, pullulan, dextran, and hyaluronic acid, poly-β-aminoesters. (Thomas et al., Molecules 2019, 24, 3744.)

In certain embodiments, the polymeric-based nanoparticles have different sizes, ranging from about 1 nm to about 1000 nm, from about 10 nm to about 500 nm, from about 50 nm to about 200 nm, from about 100 nm to about 150 nm, and from about 150 nm or less.

### I.D. Lipid Polymer Nanoparticles

Lipid polymer nanoparticles are hybrid nanoparticles providing both a lipid component and a polymer component, and as such can be considered to be an LNP or LPNP. The LPNP configuration can provide an outer polymer and inner lipid or an outer lipid and inner polymer. The presence of two different types of material facilitate designing nanoparticles providing for delayed release of a component. Different lipid and polymer components can be selected taking into account the material to be delivered (e.g., type 1 interferon receptor pathway inhibitor, cytosolic DNA-sensing inhibitor and DNA vector), along with guidance provided in herein and provided in the art. (For example, Teo et al., Advanced Drug Delivery Reviews 2016, 98, 41; Bochicchio et al., Pharmaceutics, 2021 13, 198; Mahzabin and Das, IJPSR 2021, 12(1), 65; and Teixeira et al., Progress in Lipid Research, 2018, 1.)

### I.E. Protein and Peptide-Based Nanoparticles

Protein and peptide-based systems can employ a variety of different proteins and peptides. Examples of proteins include gelatin and elastin. Peptide-based systems can employ, for example, CPPs,

CPPs are short peptides (6-30 amino acid residues) potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include cationic CPPs (highly positively charged) such as the Tat peptide, penetratin, protamine, poly-L-lysine, and polyarginine; amphipathic CPPs (chimeric or fused peptides, constructed from different sources, containing both positively and negatively charged amino acid sequences), such as transportan, VT5, bactenecin-7 (Bac7), proline-rich peptide (PPR), SAP (VRLPPP) 3, TP10, pep-1, and MPG); membranotropic CPPs (exhibit both hydrophobic and amphipathic nature simultaneously, and comprise both large aromatic residues and small residues) such as H625, SPIONs-PEG-CPP and NPs; and hydrophobic CPPs (contain only non-polar motifs or residues) such as SG3, PFVYLI, pep-7, and fibroblast growth factors.

The protein and peptide nanoparticles can be provided in different sizes for example, ranging from about 1 nm to about 1000 nm, from about 10 nm to about 500 nm, from about 50 nm to about 200 nm, from about 100 nm to about 150 nm, or from about 150 nm or less.

### I.F. Peptide Cage Nanoparticles

Peptide cage-based delivery systems can be produced from proteinaceous material able to assemble into a cage-like structure forming a constrained internal environment. Peptide cages can comprise a proteinaceous shell that self-assembles to form a protein cage (e.g., a structure with an interior cavity that is either naturally accessible to the solvent or can be made so by altering solvent concentration, pH, or equilibria ratios). The monomers of the protein cages can be naturally occurring or variant forms, including amino acid substitutions, insertions, and deletions (e.g., fragments).

Different types of protein “shells” can be assembled and loaded with different types of materials. Protein cages can be produced using viral coat protein(s) (e.g., from the Cowpea Chlorotic Mottle Virus protein coat), as well non-viral proteins (e.g., U.S. Pat. Nos. 6,180,389 and 6,984,386, U.S. Patent Application Publication No. 20040028694, and U.S. Patent Application Publication No. 20090035389, each of which is incorporated by reference herein in their entity).

Examples of protein cages derived from non-viral proteins include: eukaryotic or prokaryotic derived ferritins and apoferritins such as 12 and 24 subunit ferritins; and heat shock proteins (HSPs), such as the class of 24 subunit heat shock proteins that form an internal core space, the small HSP of Methanococcus jannaschii, the dodecameric Dsp HSP of E. coli; and the MrgA protein.

In certain embodiments, the protein cages have different core sizes, such as ranging from about 1 nm to about 1000 nm, from about 10 nm to about 500 nm, from about 50 nm to about 200 nm, from about 100 nm to about 150 nm, or from about 150 nm or less.

### I.G. Exosomes

Exosomes are small biological membrane vesicles that been utilized to deliver various cargoes including small molecules, peptides, proteins and nucleic acids. Exosomes generally range in size from about 30 nm to about 100 nm and can be taken up by a cell and deliver its cargo. Cargoes can be associated with exosome surface structure or may be encapsulated within the exosome bilayer.

Various modifications can be made to exosomes facilitating cargo delivery and cell targeting. Modifications facilitating cargo delivery include structures for associating with cargoes such as protein scaffolds and polymers. Modifications for cell targeting include targeting ligands and modifying surface charge. Publications describing production, modification, and use of exosomes for delivery of different cargoes include Munagala et al., Cancer Letters 2021, 505, 58; Fu et al., 2020 NanoImpact 20, 100261; and Dooley et al., 2021 Molecular Therapy 29(5), 1729 (each of which is hereby incorporated by reference herein).

### II. Interferon Receptor Pathway Inhibitor

The interferon pathway initiated upon interferon binding includes activation of Janus activated kinases which can phosphorylate different proteins including different signal transducer and activator of transcription (STAT) proteins. Interferons bind to the interferon receptor initiating a cascade resulting in inducing transcription of IFN-stimulated genes. Certain proteins activated by the type 1 interferon receptor pathway, such as STATI and STAT2 activate transcription of IFN-simulated genes. For example, the STAT1-STAT2-IRF9 (IFN-regulatory factor 9) complex can bind to IFN-stimulated response elements to initiate transcription; and a STAT1-STATI complex can bind to IFN-γ-activated sites to initiate transcription. There are a wide array of IFN-stimulated genes having different functions including producing protein involved in suppression of viral gene expression. (See, e.g., Yulantie et al., Acts Pharmaceutica Sinica B 2018, 86(6):889-899; Zanin et al., Frontiers in Immunology 2021, 11, article 615603; Platanias Nature Review Immunology 2005, 5:370-386; and Schoggins Annual Review of Virology 2019, 6:567-84.)

Type 1 interferon receptor (IFNAR) is made up of an interferon alpha receptor 1 subunit (IFNAR1) and an interferon alpha receptor 2 subunit (IFNAR2). Type I interferons include IFN-α (which can be further divided into different subtypes), IFN-β, IFN-δ, IFN-ε, IFN-κ, IFN-τ and IFN-ω1,2,3. IFN-α, IFN-β, IFN-e, IFN-κ and IFN-ω are present in humans. Type 1 interferon receptors can phosphorate different STATs such as STAT1 and STAT2. (Platanias Nature Review Immunology 2005, 5:370-386.)

Type 1 interferon receptor pathway inhibitors include compounds (1) that bind type 1 interferon receptor (IFNAR) and inhibit or block binding of a ligand (e.g., interferons) to the IFNAR; (2) that inhibit or block activation of the IFNAR; (3) that inhibit or block downstream activities such as Janus activated kinase (JAK) activity and signal transducer and activator of transcription (STAT) activity; that inhibit or block expression of a type 1 interferon pathway protein (e.g., IFNAR, JAK2, tyrosine kinase 1, STAT1 and/or STAT2); (4) causes or effects degradation of a Type 1 interferon pathway protein (e.g., IFNAR, JAK2, tyrosine kinase 1, STAT1 and/or STAT2); and (5) inhibiting IFNAR activation by generally inhibiting binding of ligand to IFNAR (e.g., using a decoy). In certain embodiments, the Type 1 interferon receptor pathway inhibitor is a small molecule, antibody, a polypeptide comprising an antibody fragment, peptide, nucleic acid, or a targeted protein of a degradation agent (such as a protac or degrader); or is an inhibitory nucleic acid such as a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN).

References providing examples of type 1 interferon receptor inhibitors include Gertenberger et al., J. Med. Chem. 2020, 63, 13561-13577; U.S. Pat. No. 7,465,751; U.S. Patent Application Publication No: 2022/0144957; U.S. Pat. Nos. 10,301,390; 11,136,399; 11,059,897; 10,125,195; and International Patent Publication No. WO 2006/133426.

In certain embodiments, the type 1 interferon receptor inhibitor is an antibody that binds to the type 1 interferon receptor, or comprises an antibody fragment that binds to the type 1 interferon receptor. Different types of antibodies and antibody fragments can be used including those based on IgG, those that are bispecific, human, and/or humanized. Binding fragments include FAb fragments, single chain variable region fragments (scFV), single domain fragments (dAbs), Fv fragments, camelid heavy-chain variable domains (VHHs), mini-body and diabody. Examples of antibodies and polypeptides comprising antibody fragments are provided in, for example, Strohl, Protein Cell 2018, 9(1):86-129, and Chiu et al., Antibodies 2019, 8, 55 (both of which are incorporated by reference in their entirety into the present application).

In certain embodiments, the type 1 interferon receptor inhibitor is an antibody binding to the type 1 interferon receptor; or is anifrolumab.

In certain embodiments, the type 1 interferon receptor inhibitor binds to the type 1 interferon receptor intracellularly.

In certain embodiments, the type 1 interferon receptor inhibitor binds to the type 1 interferon receptor extracellularly.

References providing JAK inhibitors, JAK inhibitor scaffolds and motifs, and design considerations include Furumoto and Gadina BioDrugs 2013, 27(5); 431-438; Hu et al., Signal Transduction and Targeted Therapy 2021, 6:402; and International Publication No. WO 2022/81872; each of these publications are hereby incorporated by reference herein in their entirety.

In different embodiments the JAK inhibitor is as provided in Table 1 or a pharmaceutically acceptable salt thereof.

TABLE 1

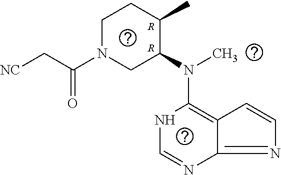

Tofacitinib

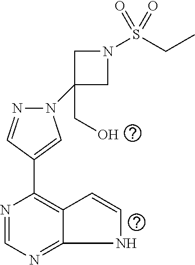 [SMILES: CCS(=O)(=O)N1CC(CO)(n2cc(-c3ncnc4[nH]ccc34)cn2)C1]

Baricitinib

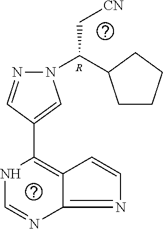

Ruxolitinib

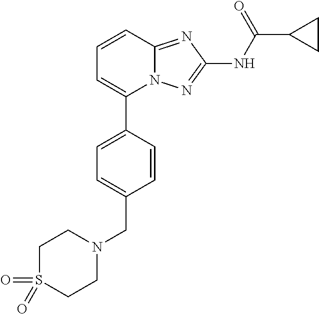 [SMILES: O=C(Nc1nc2cccc(-c3ccc(CN4CCS(=O)(=O)CC4)cc3)n2n1)C1CC1]

Filgotinib

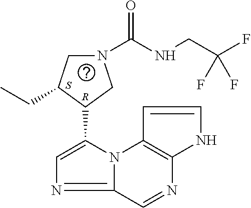

Upadacitinib

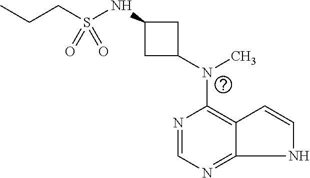

Abrocitinib

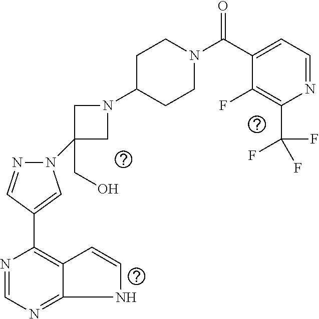 [SMILES: O=C(c1ccnc(C(F)(F)F)c1F)N1CCC(N2CC(CO)(n3cc(-c4ncnc5[nH]ccc45)cn3)C2)CC1]

Itacitinib

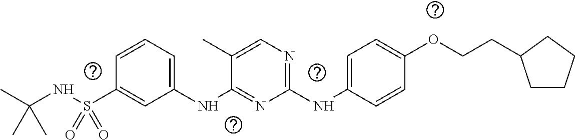 [SMILES: Cc1cnc(Nc2ccc(OCCC3CCCC3)cc2)nc1Nc1cccc(S(=O)(=O)NC(C)(C)C)c1]

Fedratinib

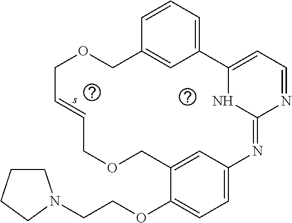 [SMILES: C1=N/C2=N/c3ccc(OCCN4CCCC4)c(c3)COC/C=C/COCc3cccc(c3)C(=C1)N2]

Pacritinib

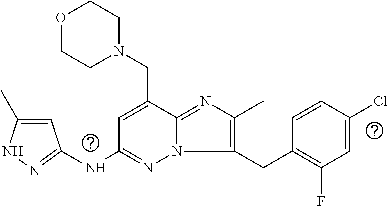 [SMILES: Cc1cc(Nc2cc(CN3CCOCC3)c3nc(C)c(Cc4ccc(Cl)cc4F)n3n2)n[nH]1]

Gandotinib

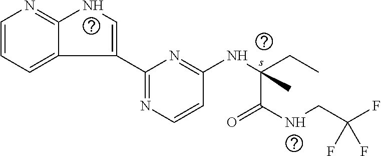 [SMILES: CC[C@@](C)(Nc1ccnc(-c2c[nH]c3ncccc23)n1)C(=O)NCC(F)(F)F]

Decernatinib

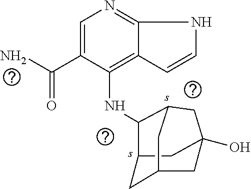 [SMILES: NC(=O)c1cnc2[nH]ccc2c1NC1C2CC3CC1CC(O)(C3)C2]

Peficitinib

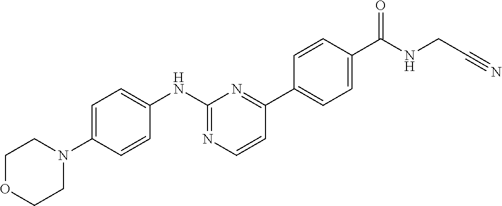 [SMILES: N#CCNC(=O)c1ccc(-c2ccnc(Nc3ccc(N4CCOCC4)cc3)n2)cc1]

Momelotinib

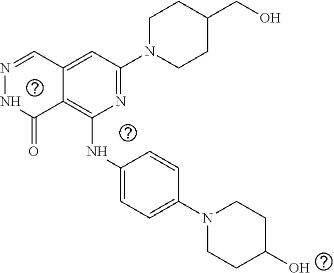 [SMILES: O=c1[nH]ncc2cc(N3CCC(CO)CC3)nc(Nc3ccc(N4CCC(O)CC4)cc3)c12]

Gusacitinib

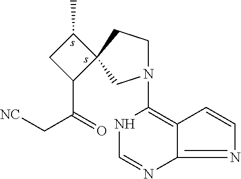 [SMILES: C[C@H]1CC(C(=O)CC#N)[C@]12CCN(c1[nH]cnc3nccc1-3)C2]

Delgocitinib

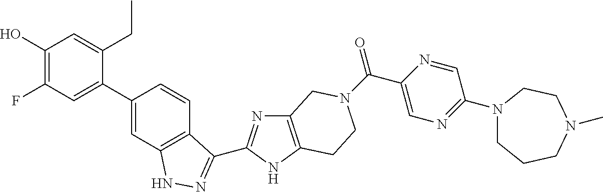 [SMILES: CCc1cc(O)c(F)cc1-c1ccc2c(-c3nc4c([nH]3)CCN(C(=O)c3cnc(N5CCCN(C)CC5)cn3)C4)n[nH]c2c1]

Compound 1

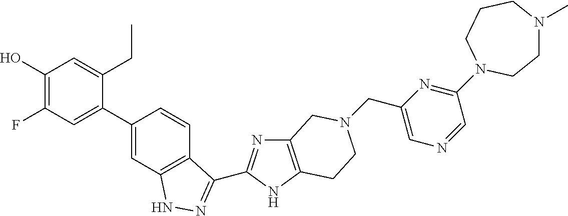 [SMILES: CCc1cc(O)c(F)cc1-c1ccc2c(-c3nc4c([nH]3)CCN(Cc3cncc(N5CCCN(C)CC5)n3)C4)n[nH]c2c1]

Compound 3

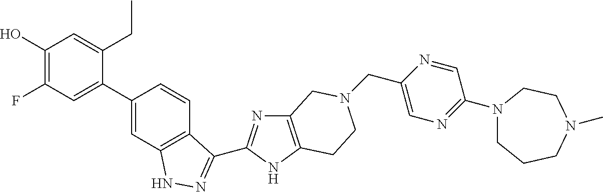 [SMILES: CCc1cc(O)c(F)cc1-c1ccc2c(-c3nc4c([nH]3)CCN(Cc3cnc(N5CCCN(C)CC5)cn3)C4)n[nH]c2c1]

Compound 2

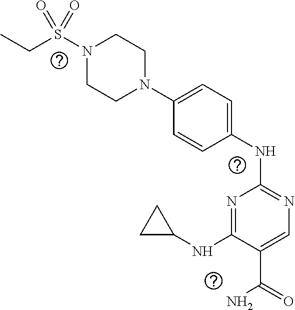 [SMILES: CCS(=O)(=O)N1CCN(c2ccc(Nc3ncc(C(N)=O)c(NC4CC4)n3)cc2)CC1]

Cerdulatinib

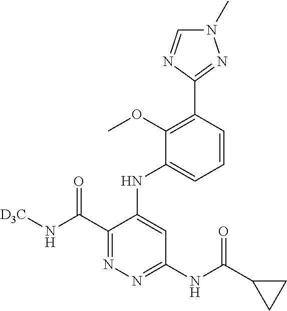 [SMILES: [2H]C([2H])([2H])NC(=O)c1nnc(NC(=O)C2CC2)cc1Nc1cccc(-c2ncn(C)n2)c1OC]

BMS-986165

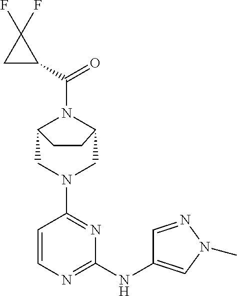 [SMILES: Cn1cc(Nc2nccc(N3C[C@H]4CC[C@@H](C3)N4C(=O)[C@@H]3CC3(F)F)n2)cn1]

PF-06700841

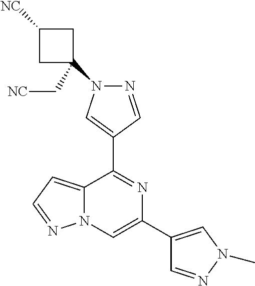

PF-06826647

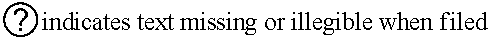

JAK inhibitors can differ in their selectively. Tofacitnib is selective for JAK1 and JAK3; baricitinib, ruxolitinib, and momelotinib are selective for JAK1 and JAK2; filgotinib, upadacitnib, abrocitinib, and itacitinib are selective for JAK 1; fedrantinib, pacritinib, and gandotinib are selective for JAK2; decernotinib and peficitinib are selective for JAK 3; gusacitinib is selective for JAK1, JAK2, JAK3 and TYK2; cerdulatinib is selective for JAK1, JAK2 and tyrosine kinase 2; Compounds 1, 2, and 3 inhibit JAK1, JAK2, JAK3 and TYK2; Compounds PF-06826647, BMS-986165, and PF-06700841 inhibit TYK2.

In different embodiments, the JAK inhibitor is selective for JAK1, is selective for JAK2, is selective for JAK3, or is selective for tyrosine kinase 2. Reference to a selective JAK inhibitor indicates the ability to significantly inhibit a particular JAK (i.e., JAK1, JAK2, JAK3, or tyrosine kinase 2) over the JAK proteins other within the group of JAK1, JAK2, JAK3, and tyrosine kinase 2. For example, an inhibitor selective for tyrosine kinase 2 inhibits tyrosine kinase 2 significantly more than it inhibits JAK1, JAK2, and JAK3. In different embodiments selective refers to a 10-fold or 100-fold difference in activity (e.g., IC₅₀).

References providing STAT inhibitors, STAT inhibitor scaffolds and motifs, and design considerations include Yulantie et al., Acta Pharmaceutica Sinica B 2018, 8(6):889-899; Hu et al., Signal Transduction and Targeted Therapy 2021, 6:402; Miklossy et al., Nat Rev Drug Discov. 2013, 12(8):611-29; and Scully et al., J. Med. Chem. 2013, 56, 4125-4129; each of these publications are hereby incorporated by reference herein in their entirety.

In different embodiments the inhibitor is STAT1 inhibitor as provided in Table 2 or a pharmaceutically acceptable salt thereof.

TABLE 2
Pravastatin

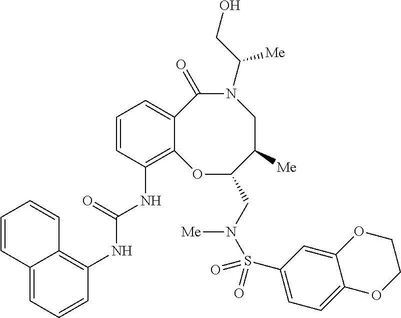 [SMILES: C[C@@H]1CN([C@@H](C)CO)C(=O)c2cccc(NC(=O)Nc3cccc4ccccc34)c2O[C@H]1CN(C)S(=O)(=O)c1ccc2c(c1)OCCO2]

BRD0476

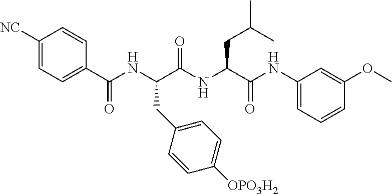

ISS840

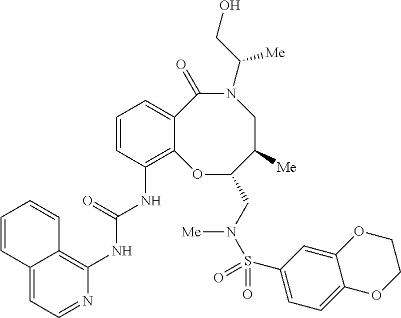 [SMILES: C[C@@H]1CN([C@@H](C)CO)C(=O)c2cccc(NC(=O)Nc3nccc4ccccc34)c2O[C@H]1CN(C)S(=O)(=O)c1ccc2c(c1)OCCO2]

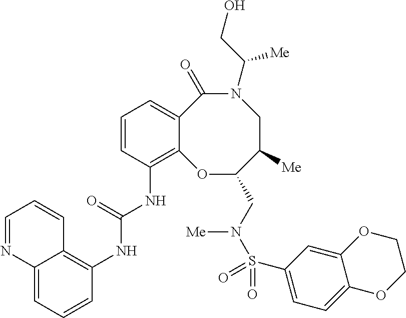 [SMILES: C[C@@H]1CN([C@@H](C)CO)C(=O)c2cccc(NC(=O)Nc3cccc4ncccc34)c2O[C@H]1CN(C)S(=O)(=O)c1ccc2c(c1)OCCO2]

### III. DNA Vector

DNA vectors comprise a transgene and one or more regulatory elements affecting RNA expression or processing from the transgene. The produced RNA can, for example, be functional or encode a particular protein. Regulatory elements may include, for example, elements modulating transcription of functional RNA, production of transgene encoded protein and protein processing. Regulatory elements that may be present include a promoter, enhancer sequences, introns, Kozak sequences, post-transcriptional regulatory elements, polyadenylation signal sequences, regulatable sequences, cell-specific regulators, and internal ribosome entry sites. Depending on the DNA vector, the vector may contain elements in addition to regulatory elements, such as terminal inverted repeats, elements facilitating plasmid replication and selection, and sequences facilitating protein secretion. Multiple transgenes which may be the same type or different; and/or multiple elements which may be of the same type or different may be present.

In an embodiment, the DNA vector is used for gene therapy. Gene therapy includes both loss-of-function and gain-of-function genetic defects. The term “loss-of-function” in reference to a genetic defect, refers to a mutation in a gene in which the protein encoded by the gene exhibits either a partial or a full loss of function that is normally associated with the wild-type protein. The term “gain-of-function” in reference to a genetic defect refers to a mutation in a gene in which the protein encoded by the gene acquires a function not normally associated with the wild type protein causes or contributes to a disease or disorder. The gain-of-function mutation can be a deletion, addition, or substitution of a nucleotide or nucleotides in the gene, giving rise to a change in the encoded protein function. In certain embodiments, the gain-of-function mutation changes the function of the mutant protein or causes interactions with other proteins. In certain embodiments, the gain-of-function mutation causes a decrease in or removal of normal wild-type protein, for example, by interaction of the altered, mutant protein with the normal wild-type protein.

Different types of DNA vectors may be employed including a minicircle, a nanoplasmid, open linear duplex DNA, closed-ended linear duplex DNA (CELiD/ceDNA/doggybone DNA), single-stranded circular DNA and single-stranded linear DNA.

In an embodiment, the DNA vector takes into account the particular mammal chosen as a subject, motifs enhancing gene expression, and sequences and motifs that induce immune stimulation. Gene expression in a particular mammal can be enhanced, for example, by codon optimization, reduction of CpG and reduction of RNA secondary structure and unstable motifs. Examples of immune stimulating motifs that be reduced include CpG, pyrimidine-rich sequences and palindrome sequences.

In different embodiments, the transgene encodes a viral antigen, a bacterial antigen, a therapeutic protein, a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN).

### III.A. Promoter

Promoters are generally located 5′ of the polynucleotide sequence being expressed and are operatively linked to the polynucleotide sequence. For example, a promoter is operatively linked with a polynucleotide sequence when it is capable of affecting the expression of sequence (e.g., the sequence is under the transcriptional control of the promoter). The promoter binds RNA polymerase and the necessary transcription factors to initiate transcription from the polynucleotide sequence. Promoter sequences define the direction of transcription and which DNA strand will be transcribed.

Encoding sequences can be operatively linked to regulatory sequences in a sense or antisense orientation. In certain embodiments, the promoter is a heterologous promoter. The term “heterologous promoter” refers to a promoter that is not found to be operatively linked to a given encoding sequence in nature.

In certain embodiments, a promoter sequence is coupled to an enhancer. Enhancers are DNA regions that increase promoter transcription. Typically, enhancers are located upstream of a promoter, but can be located downstream or within a promoter sequence. The enhancer can stimulate promoter activity and can be an innate element of the promoter or a heterologous element inserted to enhance the level or tissue-specificity of a promoter.

The promoter can be derived from different sources or produced from different elements. For example, the promoter can be entirely from a native gene, composed of different elements derived from different naturally occurring promoters, or comprise a synthetic nucleotide sequence.

Different promoters can be selected to direct the expression of a nucleotide sequence in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters are well known in the art. Examples of promoters include the phosphoglycerate kinase (PKG) promoter, CAG (composite of the CMV enhancer the chicken beta actin promoter (CBA) and the rabbit beta globin intron.), NSE (neuronal specific enolase), NeuN promoters, the SV40 early promoter, mouse mammary tumor virus LTR promoter, adenovirus major late promoter (Ad MLP), herpes simplex virus (HSV) promoter, cytomegalovirus (CMV) promoter such as the CMV immediate early promoter region (CMVIE), SFFV promoter, Rous sarcoma virus (RSV) promoter, synthetic promoters, and hybrid promoters. Other promoters can be of human origin or from other species, including mice. Common promoters include the human cytomegalovirus (CMV) immediate early gene promoter, the Rous sarcoma virus long terminal repeat, [beta]-actin, rat insulin promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter. In addition, sequences derived from non-viral genes, such as the murine metallothionein gene, may also be employed. A variety of promoter sequences are commercially available, see e.g., Stratagene (San Diego, CA).

### III.B. Additional Elements

Additional elements that may be present include an intron, an enhancer, a polyadenylation signal sequence, a Kozak sequence, a post translational regulatory element, 5′ and 3′ inverted repeats (ITRs), regulatable elements, cell-specific regulators (e.g., micoRNA binding elements), internal ribosome entry sites or other elements that affect expression or stability of the encoded sequence. or protein processing. An example of the arrangements of different elements is 5′ to 3′ promoter/enhancer, Kozak sequence, transgene, posttranscription regulatory element, and polyadenylation signal sequence. Polyadenylation signal sequences provide for the formation of a polyA tail, which facilitates nuclear export, translation and/or mRNA stability, and may also be involved in transcription termination. Examples of polyadenylation signal sequences include SV40 late polyadenylation signal, bovine growth hormone polyA (bGHpA) signal sequence, synthetic polyA, mouse β-globin pA, rabbit β-globin pA, and H4-based pA. (Buck et al., Int. J. Mol. Sci. (2020), 21, 4197).

The presence of an intron between the promoter and transgene can enhance gene expression and RNA processing. (Powell et al., Discovery Medicine 2015, 19(102), 49.) A variety of different introns can be used to enhance gene expression. Examples of introns that may be used include the rabbit β-globin intron with splice donor/splice acceptor, SV40 intron with splice donor/splice acceptor, human β-globin introns, intron 2 of the human hemoglobin beta gene, hFIX int1 (intron 1 of the human coagulation factor IX gene), CBA-rHHB (synthetic intron derived from the fusion of the intron 1 of the chicken beta actin gene and intron 2 of the rabbit hemoglobin beta), CBA (intron 1 of the chicken beta actin gene), hGH (intron 1 of the human growth hormone gene), hFIX synth (synthetic intron derived from different portions of the human coagulation factor IX gene and present in the pLIVE vector, Mirus Bio, Madison, WI); human hemoglobin subunit beta (HBB2) synthetic intron, and optimized HBB2; and chimeric introns such as introns made up of the 5′-splice donor of the first human B-globin intron and the branch and 3′-acceptor site from the intron that is between the leader and the body of the immunoglobulin gene heavy chain variable region. (Buck et al., Int. J. Mol. Sci. 2020, 21, 4197; Ronzitti et al. Mol. Ther. Methods Clin Dev. 2016 July 20; 3:16049; and the HBB-IGG intron provided by the pCMVNT™ vector.)

If desired, an encoded polypeptide can be expressed with a secretory signal sequence facilitating extracellular secretion of the polypeptide. The term “secretory signal sequence” refers to amino acid sequences functioning to enhance secretion of an operatively linked polypeptide from the cell as compared to the level of secretion seen with the polypeptide lacking the secretory signal sequence. It is not necessary that essentially all or even most of the polypeptide is secreted, as long as the secretion level is enhanced as compared with the native polypeptide. In different embodiments, at least 95%, 97%, 98%, or 99% of the polypeptide is secreted. Generally, secretory signal sequences are cleaved within the endoplasmic reticulum and may be cleaved prior to secretion. It is not necessary the secretory signal sequence is cleaved as long as secretion of the polypeptide from the cell is enhanced and the polypeptide is functional.

The secretory signal sequence can be derived in whole or in part from the secretory signal of a secreted polypeptide (i.e., from the precursor) and/or can be in whole or in part synthetic. The length of the secretory signal sequence is not critical and can be, for example, from about 10-15 to 50-60 amino acids in length. Known secretory signals from secreted polypeptides can be altered or modified (e.g., by substitution, deletion, truncation, or insertion of amino acids) as long as the resulting secretory signal sequence functions to enhance secretion of an operatively linked polypeptide. The secretory signal sequences can comprise, consist essentially of, or consist of a naturally occurring secretory signal sequence or a modification thereof. Examples of synthetic or artificial secretory signal peptides are provided in Barash et al., Biochem. Biophys. Res. Comm. 2002, 294, 835.

Kozak consensus sequences or a variation thereof play a role in translation initiation. The Kozak consensus sequence and variations are provided in, for example, McClements et al., (2021) Molecular vision, 27, 233-242.

Post-translational regulatory elements such as Woodchuck post-transcriptional regulatory element (WPRE) and Hepatitis B virus regulatory element can increase gene expression. (Buck et al., Int. J. Mol. Sci. 2020, 21, 4197, and Powell et al., Discovery Medicine 2015, 19(102), 49.)

A regulatable element can be used to increase or decrease expression. A regulatable element increasing expression of transcribed nucleic acid in response to a signal or stimuli is also referred to as an “inducible element” (i.e., is induced by a signal). Regulatable elements include tissue-specific and drug-responsive transcription (promoters/enhancers) elements. Examples of regulatable elements include tetracycline inducible elements, druggable ribozymes, druggable toe-hold switches, microRNA responsive genes (e.g., mRNA stability or protein translation), morpholino-responsive mRNAs (e.g., splicing or mRNA stability), suppressor-tRNA regulated genes, genes regulated by alternative splicing, and druggable degrons.

Typically, the amount of increase or decrease conferred by a regulatable element is proportional to the amount of signal or stimuli present. Particular examples include zinc-inducible sheep metallothionine (MT) promoter; the steroid hormone-inducible mouse mammary tumor virus (MMTV) promoter; the T7 polymerase promoter system (International Patent Publication No. WO1998/10088); the tetracycline-repressible system (Gossen, et al., Proc. Natl. Acad. Sci. USA, 1882, 89:5547-5551; the tetracycline-inducible system (Gossen et al., Science 268:1995, 1766-1769); the RU486-inducible system (Harvey et al., Curr. Opin. Chem. Biol. 1998, 2:512-518, Wang et al., Nat. Biotech. 1997, 15:239-243 and Wang et al., Gene Ther. 1997, 4:432-441; and the rapamycin-inducible system (Magari et al., J. Clin. Invest. 1997, 100:2865-2872; and Rivera et al., Nat. Medicine 1996, 2:1028-1032). Other examples of regulatable control elements include those regulated by a specific physiological state such as temperature, acute phase, or development.

### III.C. Therapeutic Proteins

DNA vectors can deliver a variety of different transgenes that can be expressed to provide a protein having a desired activity. Examples of transgenes include those providing a healthy gene copy in a subject where the native gene is defective, providing a new or modified gene that can help treat a disease or disorder, or providing a new gene encoding for protein providing a beneficial effect.

In different embodiments, a transgene encodes GAA (acid alpha-glucosidase) for treatment of Pompe disease; TPP1 (tripeptidyl peptidase-1) for treatment of late infantile neuronal ceroid lipofuscinosis type 2 (CLN2); ATP7B (copper transporting ATPase2) for treatment of Wilson's disease; alpha galactosidase for treatment of Fabry disease; ASS1 (arginosuccinate synthase) for treatment of Citrullinemia Type 1; beta-glucocerebrosidase for treatment of Gaucher disease Type 1; beta-hexosaminidase A for treatment of Tay-Sachs disease; SERPING1 (C1 protease inhibitor or C1 esterase inhibitor) for treatment of hereditary angioedema (HAE), also known as C1 inhibitor deficiency type I and type II; or glucose-6-phosphatase for treatment of glycogen storage disease type I (GSDI).

In different embodiments, the transgene encodes insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietin, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I or II (IGF-I or IGF-II), TGFß, activins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase.

In different embodiments, the transgene encodes thrombopoietin (TPO), an interleukin (IL-1 through IL-36), monocyte chemoattractant protein, leukemia inhibitory factor, granulocyte-macrophage colony stimulating factor, Fas ligand, tumor necrosis factors a or B, interferons a, B, or y, stem cell factor, flk-2/flt3 ligand, IgG, IgM, IgA, IgD or IgE, chimeric immunoglobulins, an antibody, humanized antibodies, single chain antibodies, T cell receptors, chimeric T cell receptors, single chain T cell receptors, class I or class II MHC molecules. Antibodies and immunoglobulins can, for example, be provided targeting cancer cells or other disease or disorder causing cells.

In different embodiments, the transgene encodes CFTR (cystic fibrosis transmembrane regulator protein), a blood coagulation (clotting) factor (Factor XIII, Factor IX (FIX), Factor VIII (FVIII), Factor X, Factor VII, Factor VIIa, or protein C) a gain of function blood coagulation factor, erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor a and B, a cytokine, a-interferon, β-interferon, interferon-y, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC), a peptide with immunomodulatory properties, a tolerogenic or immunogenic peptide or protein Tregitope or hCDR1, insulin, glucokinase, guanylate cyclase 2D (LCA-GUCY2D), retinal pigment epithelium-specific 65 kDa protein (RPE65), Rab escort protein 1 (choroideremia), LCA 5 (LCA-lebercilin), ornithine ketoacid aminotransferase (gyrate atrophy), retinoschisin 1 (X-linked retinoschisis), X-linked retinitis pigmentosa GTPase (XLRP), MER proto-oncogene tyrosine kinase (MERTK) (autosomal recessive (AR) forms of retinitis pigmentosa (RP)), ABCA4 (Stargardt), ACHM 2, 3 and 4 (achromatopsia), an anti-vascular endothelial growth factor (VEGF) agent polypeptide (e.g., bevacizumab, brolucizumab, ranibizumab, aflibercept), DFNB1 (connexin 26 deafness), USH1C (Usher's syndrome 1C), PKD-1 or PKD-2 (polycystic kidney disease), TPP1 (tripeptidyl peptidase-1), a sulfatase, N-acetylglucosamine-1-phosphate transferase, cathepsin A, GM2-AP, NPC1, VPC2, a sphingolipid activator protein, or one or more donor sequences used as repair templates for genome editing.

In different embodiments, the transgene encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alpha1-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

In a further embodiment the transgene encodes a protein for treating a disease or disorder selected from the group consisting of: hereditary angioedema, Pompe disease, hemophilia A, hemophilia B, Fabry, wet macular degeneration, Leber hereditary optic neuropathy, and Stargardt disease.

### III.D. Inhibitory Nucleic Acid

DNA vectors can provide a variety of different transgenes encoding for a variety of different inhibitory nucleic acid such as a short hairpin RNA (shRNA), a small interfering RNA (siRNA), a microRNA (miRNA), a RNAi, a ribozyme, and an antisense RNA. In different embodiments, the inhibitory nucleic acid binds to a gene, a transcript of a gene, or a transcript of a gene associated with a disease or disorder selected from huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Cav2.1 P/Q voltage-dependent calcium channel (CACNA1A), TATA-binding protein, Ataxin 8 opposite strand (ATXN8OS), serine/threonine-protein phosphatase 2A 55 kDa regulatory subunit B beta isoform in spinocerebellar ataxia (type 1, 2, 3, 6, 7, 8, 12 17), FMRI (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECLI also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization; caspase 2 in non-arteritic ischaemic optic neuropathy; keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection; coxsackievirus B3 genome/gene sequences in coxsackievirus B3 infection; silencing of a pathogenic allele of a gene (allele-specific silencing) like torsin A (TORIA) in primary dystonia, pan-class I and HLA-allele specific in transplant; and mutant rhodopsin gene (RHO) in autosomal dominantly inherited retinitis pigmentosa (adRP).

### III.E Gene Editing

The DNA vector can provide a variety of different transgenes encoding for a variety of different gene editing nucleic acid such as ZEN, TALEN, and CRISPR-Cas9. In different embodiments the gene editing nucleic acid edits a subject's DNA to provide a therapeutic protein as provided in Section III.C. supra., or disrupt a gene as provided in Section III.D. supra.

### IV. Cytosolic DNA-Sensing Pathway Inhibitor

The cytosolic DNA-sensing pathway detects foreign DNA and produces an immune response resulting in production of proinflammatory cytokines, proinflammatory chemokines, and Type I interferons. (See, e.g., hypertext transfer protocol://www.genome.jp/dbget-bin/www_bget?pathway+hsa04623, hereby incorporated by reference herein in its entirety.) A cytosolic DNA-sensing pathway inhibitor can be provided to reduce an immune response caused by the DNA. In different embodiments, a cGAS-STING and/or an inflammasome pathway inhibitor is employed. A particular inhibitor that inhibits more than one target can be provided as an inhibitor of each or any of the targets. Methods, compounds and compositions for inhibiting the cGAS-STING pathway and/or the inflammasome pathway are described, for example, by International Publication No. WO2023004437, hereby incorporated by reference herein in its entirety.

A variety of different types of compounds can inhibit production or activity of proteins involved in the cytosolic DNA-sensing pathway and can be used as inhibitors. In certain embodiments, the cytosolic DNA-sensing pathway inhibitor is a small molecule, antibody, peptide, inhibitory nucleic acid, or targeted protein of a degradation agent (such as a protac or degrader). Inhibitory nucleic acid can target, for example, nucleic acid encoding for a particular protein.

### IV.A. cGAS-STING Pathway Inhibitor

cGAS-STING pathway inhibitors directly affect cGAS-STING pathway proteins such as cGAS, STING, or TBK1, or can affect an agent impacting the cGAS-STING pathway. References describing cGAS-STING pathway inhibitor design, and examples of inhibitors include Ding et al., Acta Pharmaceutica Sinica B 2020, 10(12), 2272, Fu et al., iScience 2020, 23, 101026, Konno et al., Cell Rep. 2018, 23(24), 1112, U.S. Patent Application Publication No. 2020/0291001, and Haag et al., Nature, 2018, 559, 269-273; each of which are hereby incorporated by reference herein.

References providing STING inhibitors, STING inhibitor scaffolds and motifs, and design considerations include Ding et al., Acta Pharmaceutica Sinica B 2020, 10(12), 2272; Decout et al., Nat. Rev. Immunol., 2021 September; 21(9):548-569; Dubensky et al., U.S. Pat. No. 10,189,873; Katibah et al., U.S. Patent Application Publication No. 2018/0369268; Seidel et al., International Patent Publication No. WO2020/150439; Roush et al., U.S. Patent Application Publication No. 2020/0172534; Roush et al., U.S. Patent Application Publication No. 2021/236466; Glick et al., International Patent Publication No. WO 2022/140410; Hong et al., Proc Natl Acad. Sci USA., 2021 Jun. 15; 118(24); and Hong et al., Journal of Molecular Cell Biology 2022, 14(2), njac005; each of these publications are hereby incorporated by reference herein in their entirety.

In different embodiments the STING inhibitor is as provided in Table 3 or a pharmaceutically acceptable salt thereof.

TABLE 3
1-oxo-1,2,3,4-teterhydroisoquinolin-4-yl carboxylic acid
9-amino-6-chloro-2-methoxyacridine
Hydrochloroquine

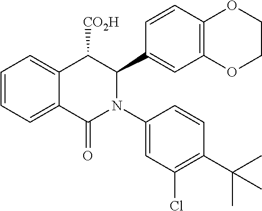 [SMILES: CC(C)(C)c1ccc(N2C(=O)c3ccccc3[C@H](C(=O)O)[C@H]2c2ccc3c(c2)OCCO3)cc1Cl]

45

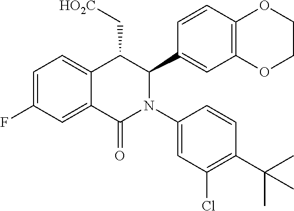 [SMILES: CC(C)(C)c1ccc(N2C(=O)c3cc(F)ccc3[C@H](CC(=O)O)[C@H]2c2ccc3c(c2)OCCO3)cc1Cl]

46

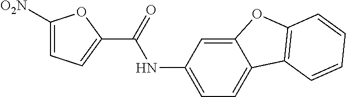 [SMILES: O=C(Nc1ccc2c(c1)oc1ccccc12)c1ccc([N+](=O)[O-])o1]

47 (C-178)

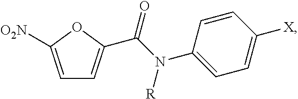

wherein R is H and X is I, R is H and
X is n-butyl or R is H and X is n-hexyl

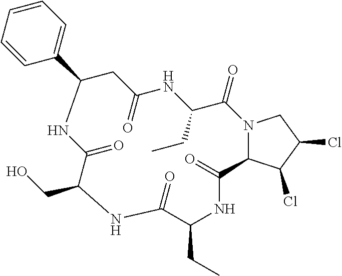 [SMILES: CC[C@@H]1NC(=O)[C@@H]2[C@H](Cl)[C@H](Cl)CN2C(=O)[C@H](CC)NC(=O)C[C@H](c2ccccc2)NC(=O)[C@H](CO)NC1=O]

53 (Astin C)

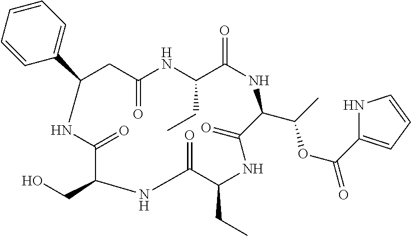 [SMILES: CC[C@@H]1NC(=O)C[C@H](c2ccccc2)NC(=O)[C@H](CO)NC(=O)[C@H](CC)NC(=O)[C@H]([C@H](C)OC(=O)c2ccc[nH]2)NC1=O]

54 (M11)

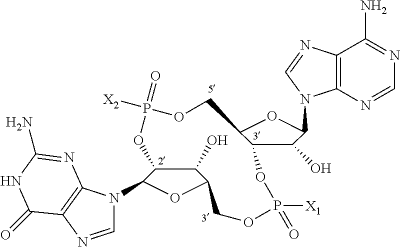

104 (2′,3′-cG*MP, X₁ = S, X₂ = O)
105 (2′,3′-cGA*MP, X₁ = O, X₂ = S)
106 (2′,3′-cG*A*MP, X₁ = S, X₂ = S)

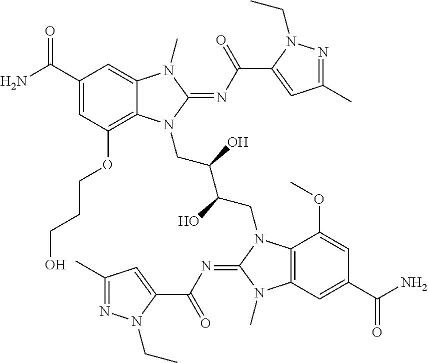 [SMILES: CCn1nc(C)cc1C(=O)/N=c1\n(C)c2cc(C(N)=O)cc(OC)c2n1C[C@@H](O)[C@H](O)Cn1/c(=N/C(=O)c2cc(C)nn2CC)n(C)c2cc(C(N)=O)cc(OCCCO)c21]

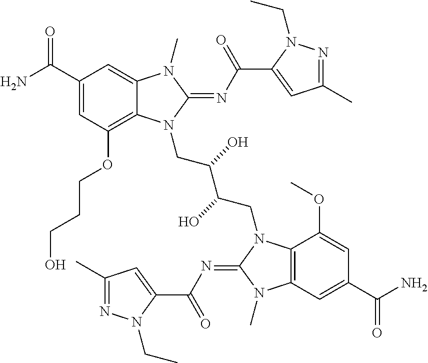 [SMILES: CCn1nc(C)cc1C(=O)/N=c1\n(C)c2cc(C(N)=O)cc(OC)c2n1C[C@H](O)[C@@H](O)Cn1/c(=N/C(=O)c2cc(C)nn2CC)n(C)c2cc(C(N)=O)cc(OCCCO)c21]

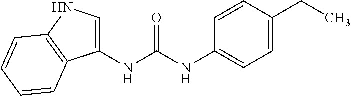 [SMILES: CCc1ccc(NC(=O)Nc2c[nH]c3ccccc23)cc1]

H-151

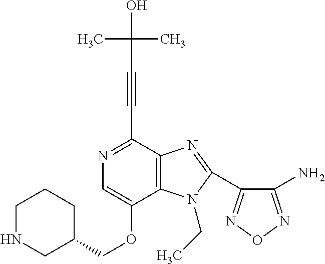 [SMILES: CCn1c(-c2nonc2N)nc2c(C#CC(C)(C)O)ncc(OC[C@H]3CCCNC3)c21]

GSK690693

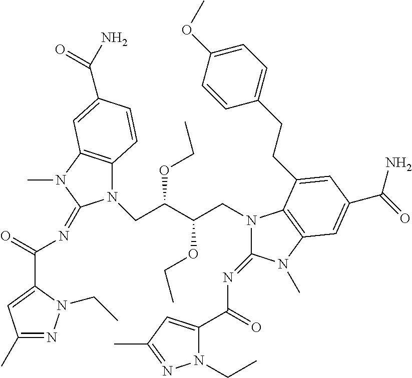 [SMILES: CCO[C@@H](Cn1/c(=N/C(=O)c2cc(C)nn2CC)n(C)c2cc(C(N)=O)ccc21)[C@H](Cn1/c(=N/C(=O)c2cc(C)nn2CC)n(C)c2cc(C(N)=O)cc(CCc3ccc(OC)cc3)c21)OCC]

(RO3015)

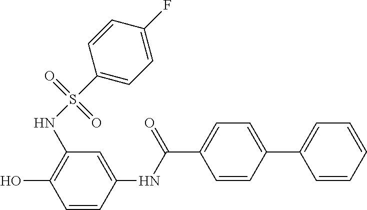 [SMILES: O=C(Nc1ccc(O)c(NS(=O)(=O)c2ccc(F)cc2)c1)c1ccc(-c2ccccc2)cc1]

SN-11

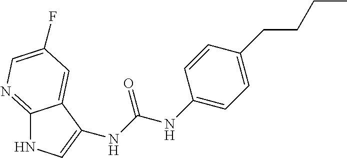 [SMILES: CCCCc1ccc(NC(=O)Nc2c[nH]c3ncc(F)cc23)cc1]

Compound 275

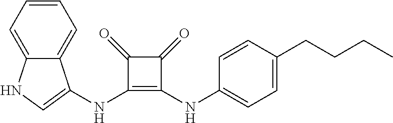 [SMILES: CCCCc1ccc(Nc2c(Nc3c[nH]c4ccccc34)c(=O)c2=O)cc1]

Compound 209

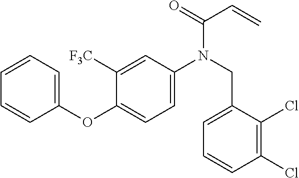 [SMILES: C=CC(=O)N(Cc1cccc(Cl)c1Cl)c1ccc(Oc2ccccc2)c(C(F)(F)F)c1]

BPK-21

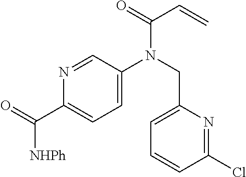

BPK-25

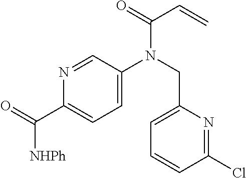

Compound 13

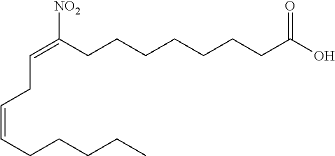 [SMILES: CCCCC/C=C\C/C=C(\CCCCCCCC(=O)O)[N+](=O)[O-]]

NO₂-cLA

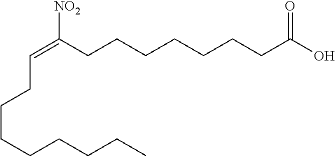 [SMILES: CCCCCCCC/C=C(\CCCCCCCC(=O)O)[N+](=O)[O-]]

NO₂-OA

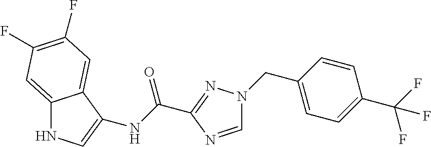 [SMILES: O=C(Nc1c[nH]c2cc(F)c(F)cc12)c1ncn(Cc2ccc(C(F)(F)F)cc2)n1]

Compound 147

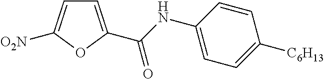

C-171

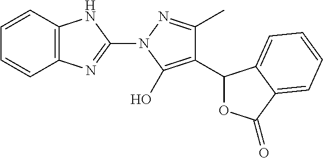 [SMILES: Cc1nn(-c2nc3ccccc3[nH]2)c(O)c1C1OC(=O)c2ccccc21]

RU-354

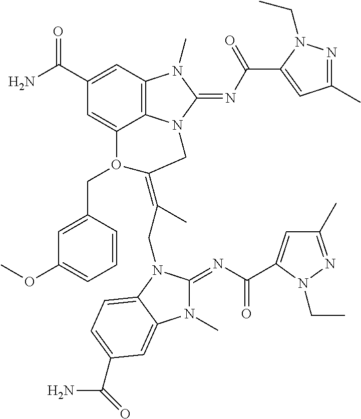

Compound 50

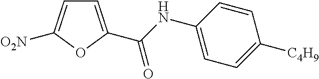

C-170

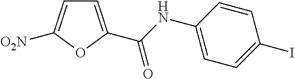 [SMILES: O=C(Nc1ccc(I)cc1)c1ccc([N+](=O)[O-])o1]

C-176

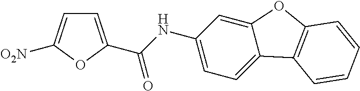 [SMILES: O=C(Nc1ccc2c(c1)oc1ccccc12)c1ccc([N+](=O)[O-])o1]

C-178

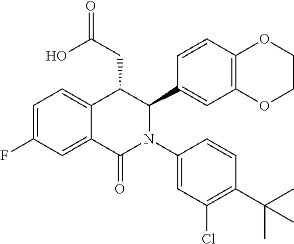 [SMILES: CC(C)(C)c1ccc(N2C(=O)c3cc(F)ccc3[C@H](CC(=O)O)[C@H]2c2ccc3c(c2)OCCO3)cc1Cl]

Compound 18

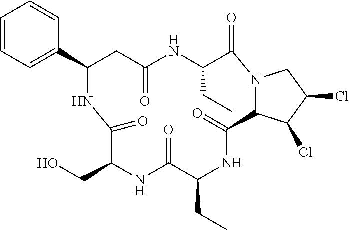

Astin C

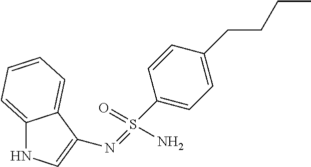 [SMILES: CCCCc1ccc(S(N)(=O)=Nc2c[nH]c3ccccc23)cc1]

Compound 118

GSK690693 is an AMP-activated proteins kinase (AMPK)/AKT inhibitor and provides an example of an inhibitor impacting the cGAS-STING pathway. AMPK/AKT activity impacts the cyclic cGAS-STING pathway by causing a loss in ULK1 phosphorylation, which releases ULK1 to phosphorylate STING thereby inhibiting STING activity. Examples of AMPK inhibitors, including GSK690693, are provided in Konno et al., Cell Rep. 2018, 23(4), 1112.

References providing cGAS inhibitors, cGAS inhibitor scaffolds and motifs, and design considerations include Ding et al., Acta Pharmaceutica Sinica B 2020, 10(12), 2272; Decout et al., Nat. Rev. Immunol., 2021 September; 21(9):548-569: Vincent et al., Nat. Commun., 2017, 8:750; Lama et al., Nat. Commun., 2019 May 21; 10(1):2261; Obioma et al., U.S. Pat. No. 10,738,056; Hong et al., Journal of Molecular Cell Biology, 2022, 14(2), njac005; Zhao et al., J. Chem. Inf. Model, 2020, 60, 3265-3276; and Padilla-Salinas et al., J. Org. Chem., 2020, 85, 1579-1600; each of these publications are hereby incorporated by reference herein in their entirety.

In different embodiments the cGAS inhibitor is as provided in Table 4 or a pharmaceutically salt thereof.

TABLE 4
quinacrine hydrochloride
ethidium bromide
actinomycin
quinacrine
9-amino-6-choloro-2-methoxyacridine
hydroxychloroquine

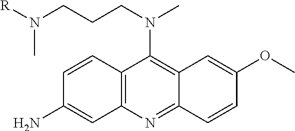

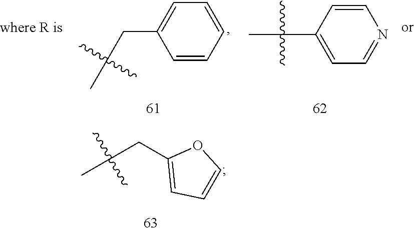 [SMILES: *Cc1ccccc1 ; *c1ccncc1 ; *Cc1ccco1]

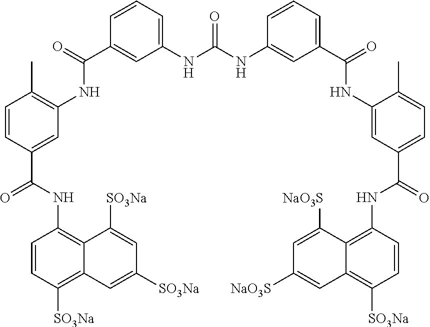

64 (Suramin)

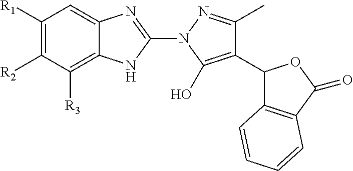

where R₁, R₂
and R₃ are each H; R₁ is H, and R₂ and R₃ are
each Cl, R₁ and R₃ are each Cl and R₂ is H; or
R₁ and R₂ are each H and R₃ is Br

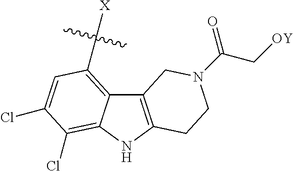

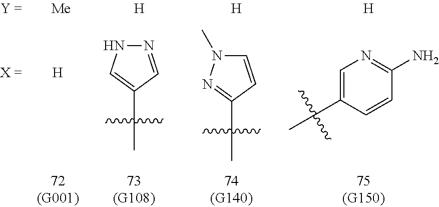

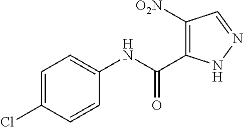 [SMILES: O=C(Nc1ccc(Cl)cc1)c1[nH]ncc1[N+](=O)[O-]]

69 (J001)

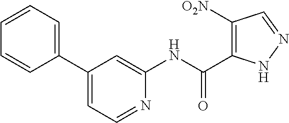 [SMILES: O=C(Nc1cc(-c2ccccc2)ccn1)c1[nH]ncc1[N+](=O)[O-]]

70 (J014)

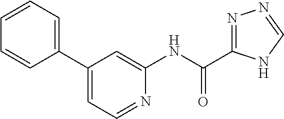 [SMILES: O=C(Nc1cc(-c2ccccc2)ccn1)c1nnc[nH]1]

71 (J073)

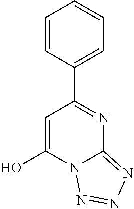 [SMILES: Oc1cc(-c2ccccc2)nc2nnnn12]

76

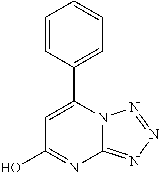 [SMILES: Oc1cc(-c2ccccc2)n2nnnc2n1]

77

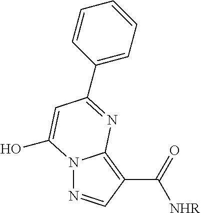

where R₁ is H or

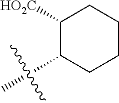

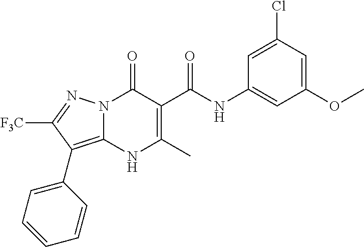 [SMILES: COc1cc(Cl)cc(NC(=O)c2c(C)[nH]c3c(-c4ccccc4)c(C(F)(F)F)nn3c2=O)c1]

81 (A1134)

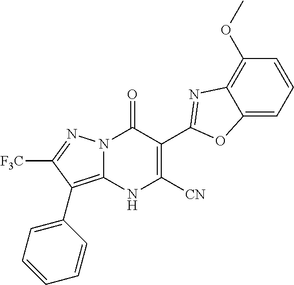 [SMILES: COc1cccc2oc(-c3c(C#N)[nH]c4c(-c5ccccc5)c(C(F)(F)F)nn4c3=O)nc12]

82 (C1069)

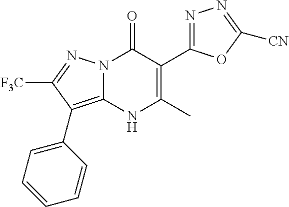 [SMILES: Cc1[nH]c2c(-c3ccccc3)c(C(F)(F)F)nn2c(=O)c1-c1nnc(C#N)o1]

83 (D1071)

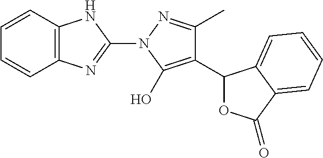 [SMILES: Cc1nn(-c2nc3ccccc3[nH]2)c(O)c1C1OC(=O)c2ccccc21]

RU-354

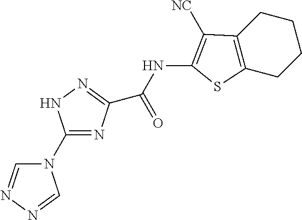 [SMILES: N#Cc1c(NC(=O)c2n[nH]c(-n3cnnc3)n2)sc2c1CCCC2]

Compound S3

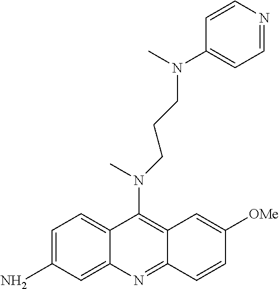 [SMILES: COc1ccc2nc3cc(N)ccc3c(N(C)CCCN(C)c3ccncc3)c2c1]

Compound X6

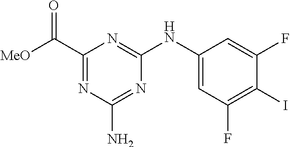 [SMILES: COC(=O)c1nc(N)nc(Nc2cc(F)c(I)c(F)c2)n1]

CU76

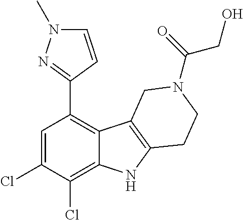 [SMILES: Cn1ccc(-c2cc(Cl)c(Cl)c3[nH]c4c(c23)CN(C(=O)CO)CC4)n1]

G140

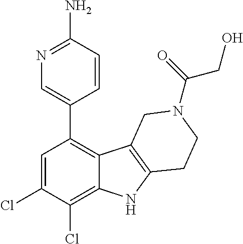 [SMILES: Nc1ccc(-c2cc(Cl)c(Cl)c3[nH]c4c(c23)CN(C(=O)CO)CC4)cn1]

G150

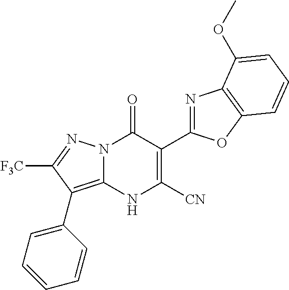 [SMILES: COc1cccc2oc(-c3c(C#N)[nH]c4c(-c5ccccc5)c(C(F)(F)F)nn4c3=O)nc12]

C1089

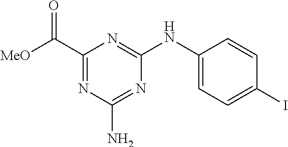 [SMILES: COC(=O)c1nc(N)nc(Nc2ccc(I)cc2)n1]

CU32

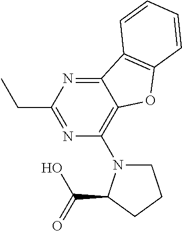 [SMILES: CCc1nc(N2CCC[C@H]2C(=O)O)c2oc3ccccc3c2n1]

Compound 15

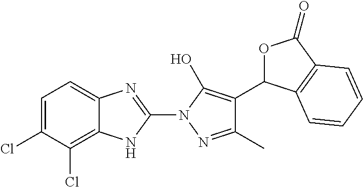 [SMILES: Cc1nn(-c2nc3ccc(Cl)c(Cl)c3[nH]2)c(O)c1C1OC(=O)c2ccccc21]

RU.521

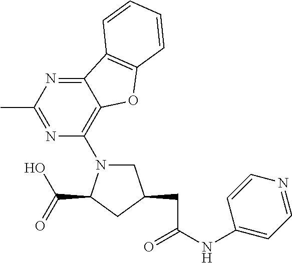 [SMILES: Cc1nc(N2C[C@@H](CC(=O)Nc3ccncc3)C[C@H]2C(=O)O)c2oc3ccccc3c2n1]

Compound 28

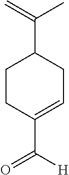

Perillaldehyde

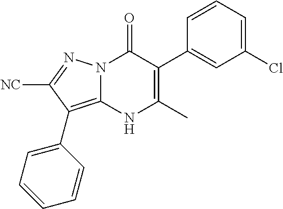 [SMILES: Cc1[nH]c2c(-c3ccccc3)c(C#N)nn2c(=O)c1-c1cccc(Cl)c1]

Compound 6

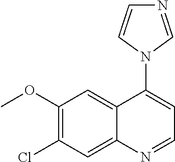 [SMILES: COc1cc2c(-n3ccnc3)ccnc2cc1Cl]

Compound 14

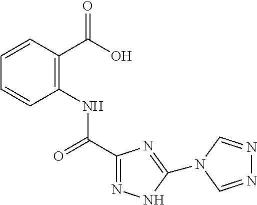 [SMILES: O=C(Nc1ccccc1C(=O)O)c1n[nH]c(-n2cnnc2)n1]

Compound 18

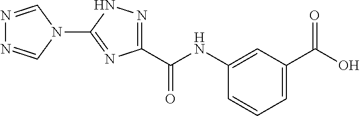 [SMILES: O=C(O)c1cccc(NC(=O)c2n[nH]c(-n3cnnc3)n2)c1]

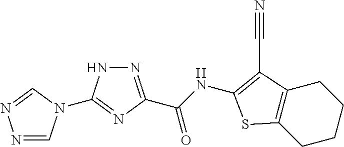 [SMILES: N#Cc1c(NC(=O)c2n[nH]c(-n3cnnc3)n2)sc2c1CCCC2]

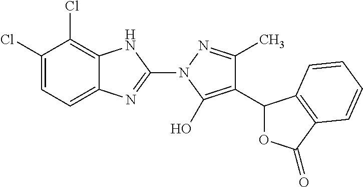 [SMILES: Cc1nn(-c2nc3ccc(Cl)c(Cl)c3[nH]2)c(O)c1C1OC(=O)c2ccccc21]

RU-521

References providing TBK1 inhibitors, TBK1 inhibitor scaffolds and motifs, and design considerations include Thomson et al., Expert Opinion on Therapeutic Patents, 2021, 31:9 785-794; Chekler et al., U.S. Application Patent Publication No. 2021/0214339; Newton and Stewart, U.S. Pat. No. 11,058,686; Karra et al., International Patent Publication No. WO 2019/079373A1; Bigi et al., U.S. Pat. No. 9,994,547; Schulze et al., U.S. Pat. No. 10,894,784; Hassan and Yan, Parmacol. Res., 2016, 111:336-342; Li et al., Int. J. Cancer 2014, 134:1972-1980; Alam et al., International Journal of Biological Macromolecules, 2022, 2027:1022-1037; Perrior et al., U.S. Pat. No. 8,962,609; and Du et al., U.S. Pat. No. 10,316,049; each of these publications are hereby incorporated by reference herein in their entirety.

In certain embodiments the TBK1 inhibitor is as provided in Table 5 or a pharmaceutically salt thereof.

TABLE 5

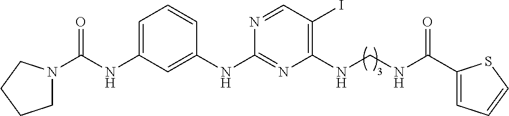

84 ( [symbol] 5)

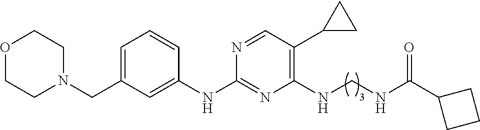

[symbol]

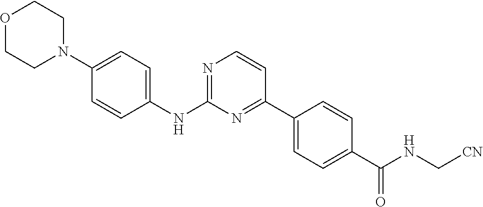 [SMILES: N#CCNC(=O)c1ccc(-c2ccnc(Nc3ccc(N4CCOCC4)cc3)n2)cc1]

86 (CYT387)

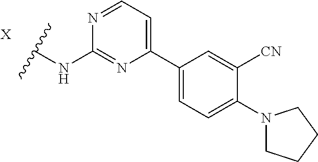

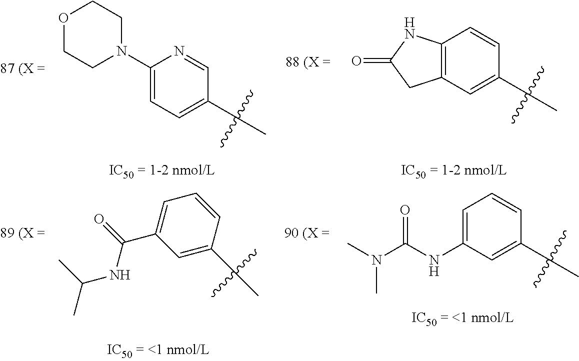 [SMILES: *c1ccc(N2CCOCC2)nc1 ; *c1ccc2c(c1)CC(=O)N2 ; *c1cccc(C(=O)NC(C)C)c1 ; *c1cccc(NC(=O)N(C)C)c1]

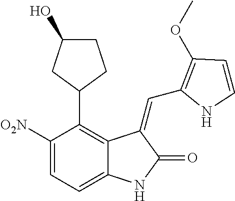 [SMILES: COc1cc[nH]c1/C=C1\C(=O)Nc2ccc([N+](=O)[O-])c(C3CC[C@H](O)C3)c21]

Oxindole 91

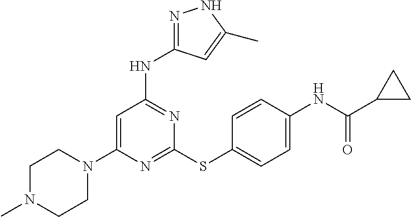 [SMILES: Cc1cc(Nc2cc(N3CCN(C)CC3)nc(Sc3ccc(NC(=O)C4CC4)cc3)n2)n[nH]1]

Tozasertib

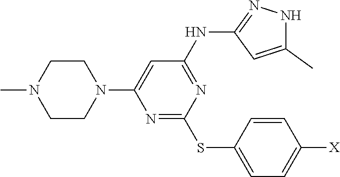

92 (X = NO₂) 93 (X = i-Pr) 94 (X = F)

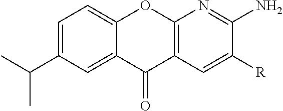

where R is COOH or

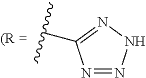

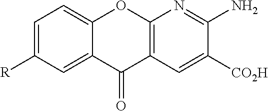

where R is either i-pentyl or chexyl,

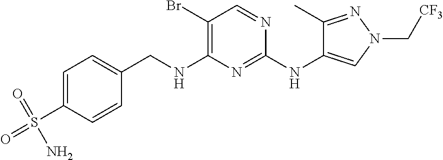 [SMILES: Cc1nn(CC(F)(F)F)cc1Nc1ncc(Br)c(NCc2ccc(S(N)(=O)=O)cc2)n1]

[symbol]

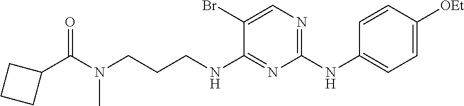 [SMILES: CCOc1ccc(Nc2ncc(Br)c(NCCCN(C)C(=O)C3CCC3)n2)cc1]

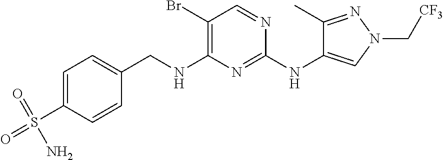 [SMILES: Cc1nn(CC(F)(F)F)cc1Nc1ncc(Br)c(NCc2ccc(S(N)(=O)=O)cc2)n1]

99 (GSK8 [symbol] 17)

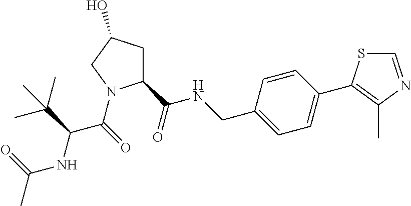 [SMILES: CC(=O)N[C@H](C(=O)N1C[C@H](O)C[C@H]1C(=O)NCc1ccc(-c2scnc2C)cc1)C(C)(C)C]

101

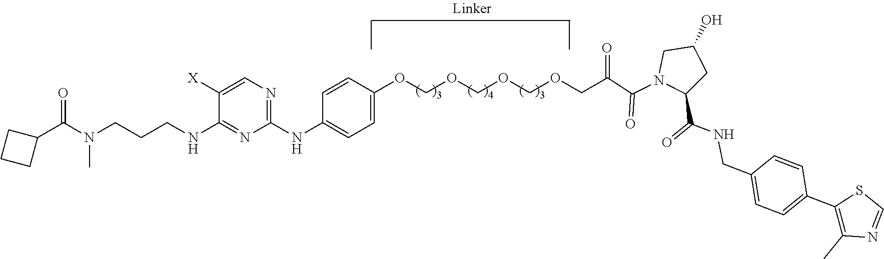

where X is either Br or I

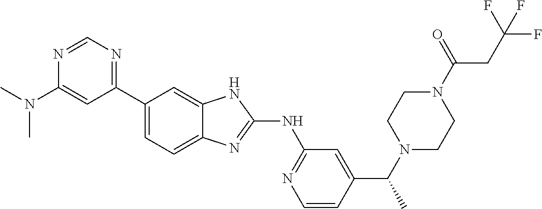 [SMILES: C[C@H](c1ccnc(Nc2nc3ccc(-c4cc(N(C)C)ncn4)cc3[nH]2)c1)N1CCN(C(=O)CC(F)(F)F)CC1]

Bay-986

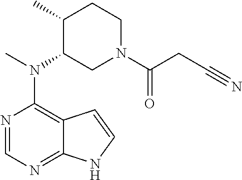 [SMILES: C[C@@H]1CCN(C(=O)CC#N)C[C@@H]1N(C)c1ncnc2[nH]ccc12]

Tofacitnib

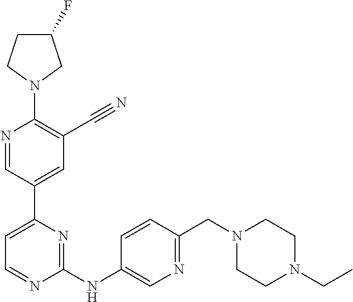 [SMILES: CCN1CCN(Cc2ccc(Nc3nccc(-c4cnc(N5CC[C@H](F)C5)c(C#N)c4)n3)cn2)CC1]

TBK-1

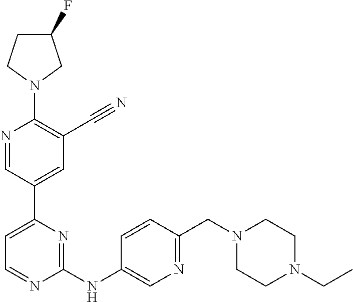 [SMILES: CCN1CCN(Cc2ccc(Nc3nccc(-c4cnc(N5CC[C@@H](F)C5)c(C#N)c4)n3)cn2)CC1]

TKB-2

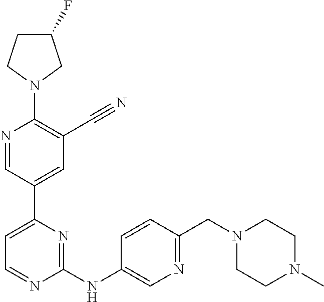 [SMILES: CN1CCN(Cc2ccc(Nc3nccc(-c4cnc(N5CC[C@H](F)C5)c(C#N)c4)n3)cn2)CC1]

TBK-3

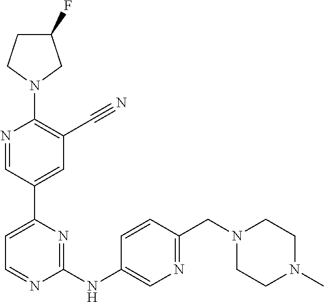 [SMILES: CN1CCN(Cc2ccc(Nc3nccc(-c4cnc(N5CC[C@@H](F)C5)c(C#N)c4)n3)cn2)CC1]

TBK-4

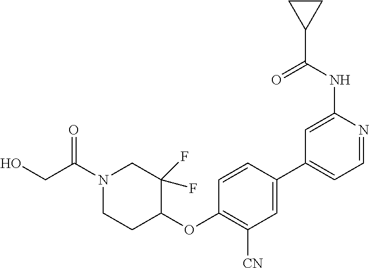 [SMILES: N#Cc1cc(-c2ccnc(NC(=O)C3CC3)c2)ccc1OC1CCN(C(=O)CO)CC1(F)F]

G-56

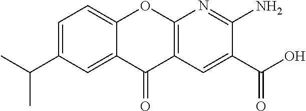 [SMILES: CC(C)c1ccc2oc3nc(N)c(C(=O)O)cc3c(=O)c2c1]

Amlexanox

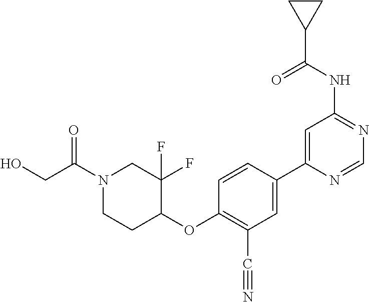 [SMILES: N#Cc1cc(-c2cc(NC(=O)C3CC3)ncn2)ccc1OC1CCN(C(=O)CO)CC1(F)F]

G-55

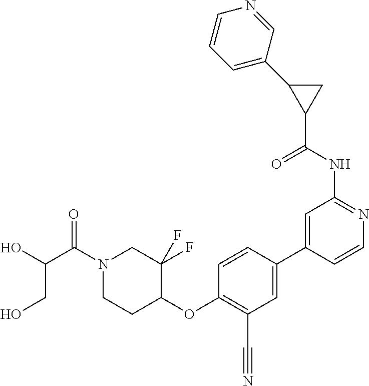 [SMILES: N#Cc1cc(-c2ccnc(NC(=O)C3CC3c3cccnc3)c2)ccc1OC1CCN(C(=O)C(O)CO)CC1(F)F]

G-59

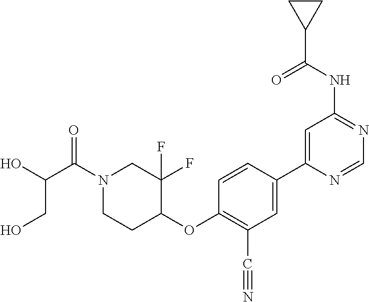 [SMILES: N#Cc1cc(-c2cc(NC(=O)C3CC3)ncn2)ccc1OC1CCN(C(=O)C(O)CO)CC1(F)F]

G-57

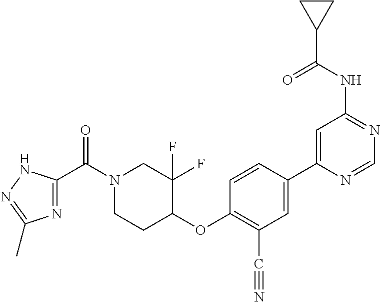 [SMILES: Cc1n[nH]c(C(=O)N2CCC(Oc3ccc(-c4cc(NC(=O)C5CC5)ncn4)cc3C#N)C(F)(F)C2)n1]

G-58

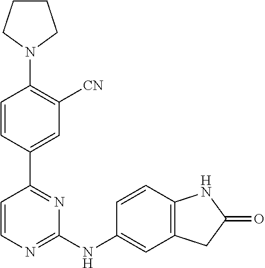 [SMILES: N#Cc1cc(-c2ccnc(Nc3ccc4c(c3)CC(=O)N4)n2)ccc1N1CCCC1]

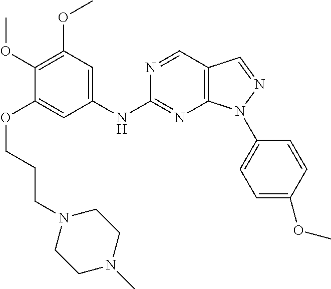 [SMILES: COc1ccc(-n2ncc3cnc(Nc4cc(OC)c(OC)c(OCCCN5CCN(C)CC5)c4)nc32)cc1]

Compound II

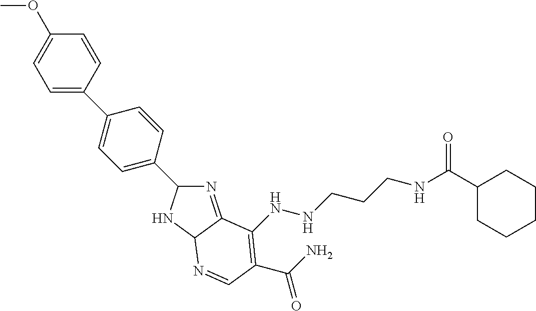 [SMILES: COc1ccc(-c2ccc(C3N=C4C(NNCCCNC(=O)C5CCCCC5)=C(C(N)=O)C=NC4N3)cc2)cc1]

AZ13102909

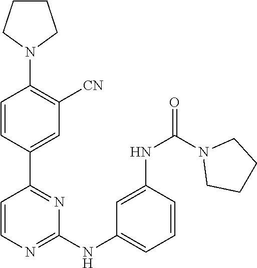 [SMILES: N#Cc1cc(-c2ccnc(Nc3cccc(NC(=O)N4CCCC4)c3)n2)ccc1N1CCCC1]

SR8185 [symbol]
indicates data missing or illegible when filed

### IV.B. Inflammasome Pathway Inhibitor

Inflammasome pathway inhibitors can directly affect an inflammasome pathway protein such as Absent in Melanoma-2 (AIM2) protein or can affect an agent impacting the inflammasome pathway. Certain DNA sequences such as the TTAGGG repeat commonly found in mammalian telomeric DNA can bind to AIM2 and suppress innate immune activation. (Kaminski et al., The Journal of Immunology, 2013, 191, 3876.) Such sequences can also inhibit other innate responses such as the cGAS and STING. Examples of AIM2 inhibitors include A151, a synthetic oligonucleotide containing four repeats of the TTAGGG motif, having the following nucleotide sequence, where the bases are joined by phosphorothioate-linkages: 5′-TTAGGGTTAGGGTTAGGGTTAGGG-3′ (SEQ ID NO: 1); and 5′-TTAGGGTTAGGGTTAGGGTTAGGG-3′ (SEQ ID NO: 2), containing phosphodiester linkages. Additional oligonucleotide sequences include other types of modified SEQ ID NO: 2, such as those having the same nucleotide sequence, but different modified backbones. Such nucleotide sequences can also be used as cGAS and STING inhibitors and can be incorporated into the DNA or DNA vector.

### V. Immune Cell Modulators

In some cases, administration of the DNA vector can result in an undesirable immune response due to, for example, DNA vector components, the transgene product being recognized as foreign, or an edited gene producing a protein being regarded as foreign. In such cases, if desired, the host immune response can be reduced, for example using an immune cell modulator or immunosuppressant. In some cases, such as cancer treatment, viral treatment and bacterial treatment certain responses may be advantageous.

### V.A. Phagocyte-Depleting Agent

In certain embodiments a phagocyte-depleting agent is used. Methods, compounds and compositions for depletion of phagocytic immune cells are described, for example, by International Publication No. WO2022140788A1, hereby incorporated by reference herein in its entirety.

A “phagocyte-depleting agent” refers to an agent that depletes or destroys phagocytes in a subject and/or interferes with one or more phagocyte functions. Phagocytes, also referred to herein as phagocytic cells, phagocytic immune cells, phagocyte cells, or phagocyte immune cells, include macrophages, monocytes, neutrophils, and dendritic cells. Langerhans cells are dendritic cells found in the skin. Mast cells are found in many tissues including lung or skin and can also act as phagocytes.

A “monocyte and/or macrophage-depleting agent” refers to an agent that depletes or destroys monocytes and/or macrophages in a subject and/or interferes with one or more monocyte and/or macrophage functions. The monocyte and/or macrophage-depleting agents can target monocytes and/or macrophages. Macrophages are mononuclear phagocytes that are differentiated monocytes. In different tissues, macrophages are referred to by different names. Examples of tissue-specific, or resident, macrophages include Kupffer cells in the liver, intestinal macrophages in the gut, microglial cells in the brain, alveolar macrophages in the lung, resident kidney macrophages, skin macrophages, red pulp macrophages in the spleen, and osteoclasts in bone. Examples of monocyte- and/or macrophage-depleting agents include agents targeting phagocytic immune cell markers, e.g., CD115 inhibiting agents such as anti-CD115 antibodies or CD115 small molecule inhibitors; F4/80 inhibiting agents such as anti-F4/80 antibodies or F4/80 small molecule inhibitors; CD68 inhibiting agents such as anti-CD68 antibodies or CD68 small molecule inhibitors; CD11b inhibiting agents such as anti-CD11b antibodies or CD11b small molecule inhibitors; the chemotherapeutic agent Trabectedin; intralipids; empty liposomes; and bisphosphonates including clodronate. In certain embodiments, the monocyte and/or macrophage-depleting agent is not clodronate. In certain embodiments, clodronate and at least one additional monocyte and/or macrophage-depleting agent are used together.

A “neutrophil-depleting agent” refers to an agent that depletes or destroys neutrophils in a subject and/or interferes with one or more neutrophil functions. The neutrophil-depleting agents target neutrophils. Examples of neutrophil-depleting agents include agents targeting phagocytic immune cell markers such Ly6G inhibiting agents, including anti-Ly6G antibodies or Ly6G small molecule inhibitors; CD177 inhibiting agents including anti-CD177 antibodies or CD177 small molecule inhibitors; CD14 inhibiting agents including anti-CD14 antibodies or CD14 small molecule inhibitors; CD15 inhibiting agents including anti-CD15 antibodies or CD15 small molecule inhibitors; CD11b inhibiting agents including anti-CD11b antibodies or CD11b small molecule inhibitors; CD16 inhibiting agents, including anti-CD16 antibodies or CD16 small molecule inhibitors; CD32 inhibiting agents including anti-CD32 antibodies or CD32 small molecule inhibitors; CD33 inhibiting agents including anti-CD33 antibodies or CD33 small molecule inhibitors; CD44 inhibiting agents including anti-CD44 antibodies or CD44 small molecule inhibitors; CD45 inhibiting agents including anti-CD45 antibodies or CD45 small molecule inhibitors; CD66b inhibiting agents including anti-CD66b antibodies or CD66b small molecule inhibitors; CD18, or inhibiting agents including anti-CD18 antibodies or CD18 small molecule inhibitors; CD62L inhibiting agents including anti-CD62L antibodies or CD62L small molecule inhibitors; and Gr-1 inhibiting agents anti-Gr-1 antibodies or Gr-1 small molecule inhibitors.

A “dendritic cell-depleting agent” refers to an agent that depletes or destroys dendritic cells in a subject and/or interferes with one or more dendrite functions. The dendritic cell-depleting agents can target any dendritic cell. Examples of dendritic cell-depleting agents include agents that target phagocytic immune cell markers such as PDCA1 inhibiting agents, including anti-PDCA1 antibodies or PDCA1 small molecule inhibitors; and CD11c inhibiting agents including anti-CD11c antibodies or CD11c small molecule inhibitors.

A “inhibiting agent” refers to any compound capable of down-regulating, decreasing, reducing, suppressing, or inactivating the amount and/or activity of the targeted protein. Inhibiting agents can be proteins, oligo- and polypeptides, nucleic acids, genes, or chemical molecules. Suitable protein inhibitors can be, for example, monoclonal or polyclonal antibodies which bind to the targeted protein; and small molecules.

Examples of CD115 inhibiting agent include CD115 small molecule inhibitors pexidartinib (PLX-3397), BLZ-945, Linifanib (ABT-869), JNJ-28312141 (Johnson & Johnson), JNJ-40346527 (Johnson & Johnson), PLX7486 (Plexxikon), ARRY-382 (Array BioPharma), anti-CD115 antibody such as AFS98 (Invitrogen or BioCell), 12-3A3-1B10 (Invitrogen), 6C7 (Bioss), Cabiralizumab (FPA008), 25949-1-AP (Proteintech), 1G4 (Abnova), 3G12 (Abnova), 604B5 2E11 (Invitrogen), Emactuzumab (RG-7155; Roche), AMG 820 (Amgen), IMC-CS4, and ROS8G11 (Invitrogen). In certain embodiments, the antibody or antigen-binding fragment thereof is AFS98 (e.g., BioCell BE0213 and Oncogene 1995; 11(12):2469-2476).

Suitable Ly6G inhibiting agent, including those known to those skilled in the art, in view of the present disclosure can be used. Examples of anti-Ly6G antibodies include A8 (BioCell BP0075-1) and RB6-8C5 (ab25377).

Intralipid and empty liposomes have been shown to interfere with one or more functions of monocytes and/or macrophages. See, e.g., Liu et al., Biochim Biophys Acta. 2013c, June; 1830(6):3447-53 and Saunders et al., Nano Lett. 2020 Jun. 10; 20(6):4264-4269. Pretreatment with intralipid or empty liposomes can effectively saturate monocyte/macrophage cells and prevent phagocytosis of a non-viral therapeutic agent. Examples of intralipids and empty liposomes include I141-100ML (Sigma Aldrich), 2B6063 (Baxter), and those described in Liu et al., Biochim Biophys Acta. 2013 June; 1830(6):3447-53 and Saunders et al., Nano Lett. 2020 Jun. 10; 20(6):4264-4269.

Examples of bisphosphonates include clodronate, pamidronate, ibandronate, alendronate, and zoledronate.

Other examples of “phagocyte-depleting agent” include palbociclib (Ibrance®; Pfizer), and cromolyn sodium (Nasalcrom®; Bausch & Lomb).

### V.B. Immunosuppressant

An immunosuppressant is a compound capable of slowing or halting immune system activity in a subject. A variety of different immune responses can be produced including innate immune responses and humoral immune responses. For example, immune responses include a detectable alteration in Toll receptor activation, lymphokine (e.g., cytokine or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion of cells of the immune system and increased processing and presentation of antigen by antigen presenting cells.

Examples of immunosuppressants include a calcineurin inhibitor, such as cyclosporine, ISA (TX) 247, tacrolimus or calcineurin; a target of rapamycin such as sirolimus, everolimus, FK778 or TAFA-93; interleukin-2 a-chain blocker such as basiliximab and daclizumab; inosine monophosphate dehydrogenase inhibitor, such as mycophenolate mofetil; dihydrofolic acid reductase inhibitor such as methotrexate; immunosuppressive antimetabolite such as azathioprine; cytokine inhibitors such as an anti-cytokine antibody, e.g., Siltuximab; or a steroid.

In certain embodiments, an immunosuppressant is an anti-inflammatory agent. In certain embodiments, an immunosuppressant is a steroid, e.g., a corticosteroid, prednisone, prednisolone, cyclosporine (e.g., cyclosporine A), mycophenolate; a B cell targeting antibody, e.g., rituximab; a proteasome inhibitor, e.g., bortezomib; a mammalian target of rapamycin (mTOR) inhibitor, e.g., rapamycin; a tyrosine kinase inhibitor, e.g., ibrutinib; an inhibitor of B-cell activating factor (BAFF); or an inhibitor of a proliferation-inducing ligand (APRIL) or a derivative thereof. In certain embodiments, the immunosuppressive agent is an anti-IL-1β agent (e.g., anti-IL-1β monoclonal antibody canakinumab (Ilaris®)) or an anti-IL-6 agent (e.g., anti-IL-6 antibody sirukumab or anti-IL-6 receptor antibody tocilizumab (Actemra®)), or a combination thereof.

The term “steroid” refers to a chemical substance comprising three cyclohexane rings and a cyclopentane ring. The rings are arranged to form tetracyclic cyclopentaphenanthrene, i.e., gonane. There are different types of steroids such as corticosteroids and glucocorticosteroids.

The term “corticosteroid” refers to a class of steroid hormones produced in the adrenal cortex or produced synthetically. In certain embodiments, the steroid can be a corticosteroid. Corticosteroids are involved in a wide range of physiologic systems such as stress response, immune response and regulation of inflammation, carbohydrate metabolism, protein catabolismblood electrolyte levels, and behavior. Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Non-limiting examples of corticosteroids include, aldosternone, beclomethasone, beclomethasone dipropionate, betametahasone, betametahasone-21-phosphate disodium, betametahasone valerate, budesonide, clobetasol, clobetasol propionate, clobetasone butyrate, clocortolone pivalate, cortisol, cortisteron, cortisone, deflazacort, dexamethasone, dexamethasone acetate, dexamethasone sodium phosphate, diflorasone diacetate, dihydroxycortison, flucinonide, fludrocortisones acetate, flumethasone, flunisolide, flucionolone acetonide, fluticasone furate, fluticasone propionate, halcinonide, halpmetasone, hydrocortisone, hydrocortisone acetate, hydrocortisone succinate, 16a-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate.

A variety of generic and brand name corticosteroids are available including: cortisone (CORTONE™ ACETATE™, ADRESON™, ALTESONA™, CORTELANT™, CORTISTAB™, CORTISYL™, CORTOGEN™, CORTONE™, SCHEROSON™); dexamethasone-oral (DECADRON ORAL™, DEXAMETH™, DEXONE™, HEXADROL-ORAL™, DEXAMETHASONE™ INTENSOL™, DEXONE 0.5™, DEXONE 0.75™, DEXONE 1.5™, DEXONE 4™); hydrocortisone-oral (CORTEF™, HYDROCORTONE™); hydrocortisone cypionate (CORTEF ORAL SUSPENSION™); methylprednisolone-oral (MEDROL-ORAL™); prednisolone-oral (PRELONE™, DELTA-CORTEF™, PEDIAPRED™, ADNISOLONE™, CORTALONE™, DELTACORTRIL™, DELTASOLONE™, DELTASTAB™, DI-ADRESON FTM, ENCORTOLONE™, HYDROCORTANCYL™, MEDISOLONE™, METICORTELONE™, OPREDSONE™, PANAAFCORTELONE™, PRECORTISYL™, PRENISOLONA™, SCHERISOLONA™ SCHERISOLONE™); prednisone (DELTASONE™, LIQUID PRED™, METICORTENT™, ORASONE 1™, ORASONE 5™, ORASONE 10™, ORASONE 20TM ORASONE 50™, PREDNICEN-M™, PREDNISONE INTENSOL™, STERAPRED™, STERAPRED DS™, ADASONE™, CARTANCYL™, COLISONE™, CORDROL™, CORTAN™, DACORTIN™, DECORTIN™, DECORTISYL™, DELCORTIN™, DELLACORT™, DELTADOME™, DELTACORTENE™, DELTISONA™, DIADRESON™, ECONOSONE™, ENCORTON™, FERNISONE™, NISONA™, NOVOPREDNISONE™, PANAFCORT™, PANASOL™, PARACORT™, PARMENISON™, PEHACORT™, PREDELTIN™, PREDNICORT™, PREDNICOT™, PREDNIDIB™, PREDNIMENT™, RECTODELT™, ULTRACORTEN™, WINPRED™); triamcinoloneoral (KENACORT™, ARISTOCORT™, ATOLONE™, SHOLOG ATM, TRAMACORT-DTM, TRI-MED™, TRIAMCOT™, TRISTOPLEX™, TRYLONE DTM, U-TRI-LONE™). In certain embodiments, a corticosteroid can be dexamethasone, prednisone, prednisolone, triamcinolone, clobetasol propionate, betamethasone valerate, betamethasone dipropionate, or mometasone furoate. Methods of synthesizing steroids and corticosteroids are well known in the art and many are also commercially available.

A corticosteroid such as dexamethasone can be delivered, for example, as free dexamethasone, as a separate LNP composition or as part of the same LNP composition as the DNA. Chen et al., Journal of Controlled Release 2018, 286, 46-54, includes a description of LNP providing nucleic acid and dexamethasone linked to fatty acid.

### VI. Pharmaceutical Compositions

A pharmaceutical composition contains one or more active component along with a pharmaceutical acceptable carrier. Reference to “pharmaceutically” or “pharmaceutically acceptable” refers to non-toxic molecular entities suitable for administration and/or storage. Pharmaceutical compositions can comprise more than one therapeutically active agent. Examples of pharmaceutically acceptable carriers include a non-toxic (in the amount used) solid, semi-solid or liquid filler, diluent, encapsulating material, or formulation.

The form of the pharmaceutical compositions, the route of administration, the dosage and the regimen depend upon the condition to be treated, such as the severity of the illness, the age, weight, and sex of the patient. Pharmaceutical compositions for the agents described herein can be formulated for topical, oral, intranasal, parenteral, intraocular, intravenous, intramuscular, or subcutaneous administration.

In an embodiment, the pharmaceutical composition contains a formulation capable of injection into a subject. Examples of injectable formulation components include isotonic, sterile, saline solutions (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition, depending on the case, of sterilized water or physiological saline, permit the constitution of injectable solutions. The doses used for the administration can be adapted as a function of various parameters such as mode of administration, relevant pathology, and duration of treatment.

Other pharmaceutically acceptable forms include tablets or other solids for oral administration, including time release capsules.

A pharmaceutical composition comprising a DNA vector comprising a therapeutic transgene can be administered to a subject at dose suitable for treating a particular disease or disorder. In different embodiments a suitable dosage can be from about 0.01 mg/kg to about 10 mg/kg of vector per kg body weight of a subject, about 0.01 mg/kg to about 0.1 mg/kg of vector per kg body weight of a subject, about 0.1 mg/kg to about 1.0 mg/kg of vector per kg body weight of a subject, and about 1.0 mg/kg to about 10 mg/kg of vector per body weight of a subject.

A small molecule cytosolic DNA-sensing inhibitor, type 1 interferon receptor pathway inhibitor, or immune cell modulator inhibitor can be administered to a subject at a suitable dose taking into account the DNA vector and disease or disorder being treated. In different embodiments a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

An antibody that targets a type 1 interferon receptor can be administered to a subject at a suitable dose taking into account the DNA vector and disease or disorder being treated. For example, a suitable dosage can be from about 0.01 mg/kg to about 5 mg/kg body weight of a subject, wherein the dosage is administered in 1 to 10 total injections.

An antibody that targets a phagocytic immune cell marker can be administered to a subject at a suitable dose. For example, a suitable dosage can be from about 0.01 mg/kg to about 5 mg/kg body weight of a subject, wherein the dosage is administered in 1 to 10 total injections.

A CD115 inhibitor such as pexidartinib can be administered to a subject at a suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

A bisphosphonate, for example, clodronate, can be administered to a subject at a suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

A corticosteroid, e.g., dexamethasone, can be administered to a subject at a suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

Various compounds described herein can be provided as a pharmaceutically acceptable salt. Reference to a “pharmaceutically acceptable salt” indicates to a salt suitable for administration to a subject. Depending on the compound, pharmaceutically acceptable salts include acid addition and basic salts. Pharmaceutically acceptable acid addition salts include hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include aluminum, calcium, lithium, magnesium, potassium, sodium, zinc, bismuth, and diethanolamine salts. In addition, various amino acids can be employed as pharmaceutically acceptable salts.

### VII. Administration and Treatment

The different compounds and compositions described herein can be administered to a subject for different purposes including research purposes and to treat a disease or disorder in a mammal. Preferred uses are to treat a disease of disorder in a human.

Reference to “treatment” or “treat” refers to both prophylactic, and therapeutic treatment of a patient having a disease or disorder. Reference to “prophylactic” treatment indicates a decrease in the likelihood of contracting a disease or disorder or decreasing the potential severity of a disease or disorder. Reference to “therapeutic” indicates a clinical meaningful amelioration in at least one symptom or cause associated with a disease or disorder. Thus, treatments include administration to subjects at risk of contracting the disease or disorder, suspected to have contracted the disease or disorder, as well as subjects who are ill or have been diagnosed as suffering from a disease or disorder and includes suppression of clinical relapse.

The terms “ameliorate”, and “amelioration” refer to a detectable or measurable improvement in a disease or disorder symptom or an underlying cellular response. A detectable or measurable improvement includes a subjective or objective decrease, reduction, inhibition, suppression, limit or control in the occurrence, frequency, severity, progression, or duration of the disease or disorder, or complication caused by or associated with the disease or disorder, or an improvement in a symptom or an underlying cause or a consequence of the disease or disorder, or a reversal of the disease or disorder. For Pompe, an effective amount includes an amount that inhibits or reduces glycogen production or accumulation, enhances, or increases glycogen degradation or removal, improves muscle tone and/or muscle strength and/or respiratory function. For HemA or HemB, an effective amount includes an amount that reduces frequency or severity of acute bleeding episodes in a subject and an amount that reduces clotting time as measured by a clotting assay.

The terms “effective amount” and “sufficient amount” is that amount required to obtain a desired effect. Treatment can be carried out by administering a therapeutically effective amount of DNA vector to a subject. A therapeutically effective amount can be provided in single or multiple doses to achieve a therapeutic or prophylactic effect.

Different agents can be administered in an “effective amount” or “sufficient amount” to achieve the desired effect. For example, an effective amount of a type 1 interferon receptor pathway inhibitor is an amount provided in single or multiple doses that inhibits the activity of the type 1 interferon receptor pathway resulting in a decrease in one or more activities of the innate immune response in response to DNA; inhibits transcriptional activation of one or more interferon stimulated genes; and/or increases the tolerability of DNA vector administration. In different embodiments the amount is effective to reduce cytokine product stimulated by DNA; or reduce IL-6, IFN alpha, and/or IFN gamma expression induced by DNA administration.

In different embodiments, an effective amount the GAS-STING pathway inhibitor or the inflammasome pathway inhibitor is the amount provided in single or multiple doses that inhibits the activity of the cGAS-STING pathway or the inflammasome pathway, and provides a decrease in one or more activities of the innate immune response; an effective amount of immune cell modulators is that amount provided in single or multiple doses that provides a detectable reduction in phagocytes and/or phagocyte function; and an effective amount of immunosuppressant is that amount provided in single or multiple doses that inhibits an immune system activity.

An effective amount can be administered alone or can be administered in combination with another composition, treatment, protocol, or therapeutic regimen. The amount can be proportionally increased, for example, based on the need of the treatment, subject, type, status and severity of the disease or disorder treated or side effects.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The DNA vector and the type 1 interferon receptor pathway inhibitor when separately administered can be provided in any order or at approximately the same time. In certain embodiments, the inhibitor is administered at least 60 minutes, at least 90 minutes, or at least 120 minutes prior to the DNA vector.

In certain embodiments the type 1 interferon receptor pathway inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, up to about 4 hours, up to about 5 hours, up to about 6 hours, up to about 7 hours, up to about 8 hours, up to about 9 hours, up to about 10 hours, up to about 12 hours, up to about 1 day, up to about 2 days, up to about 3 days, up to about 4 days, or up to about a week prior to DNA vector administration. In certain embodiments, the type 1 interferon receptor pathway inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, or about 4 hours prior to DNA vector administration. In certain embodiments the type 1 interferon receptor pathway inhibitor is administered about 15 minutes, about 30 minutes, about 45 minutes, about 60 minutes, about 90 minutes, about 120 minutes, or about 1 day after the DNA vector.

In certain embodiments type 1 interferon receptor pathway inhibitor is administered at about the same time, about 5 minutes, about 10 minutes, about 15 minutes, about 30 minutes, about 45 minutes, about 60 minutes, about 90 minutes, about 2 hours, about 2.5 hours, about 3 hours, about 3.5 hours, or about 4 hours prior to DNA vector administration.

In certain embodiments, the DNA vector and the type 1 interferon receptor pathway inhibitor can be provided in the same nanoparticle to release at about the same time, or the nanoparticle can be designed to delay release of one of the components. In an embodiment, the nanoparticle is designed to release the inhibitor prior to the release of the DNA vector.

In certain embodiment involving the use of a cytosolic DNA-sensing inhibitor, the cytosolic DNA-sensing inhibitor when separately administered can be provided in any order or at approximately the same time as the type 1 interferon receptor pathway inhibitor and DNA vector. In certain embodiments, the cytosolic DNA-sensing inhibitor is independently (with respect to the type 1 interferon receptor pathway inhibitor) administered at least 60 minutes, at least 90 minutes, or at least 120 minutes prior to the DNA vector.

In certain embodiments the cytosolic DNA-sensing inhibitor is administered independently (with respect to the type 1 interferon receptor pathway inhibitor) at about the same time, up to about 5 minutes, up to about 15 minutes, up to about 30 minutes, up to about 45 minutes, up to about 60 minutes, up to about 90 minutes, up to about 2 hours, up to about 3 hours, up to about 4 hours, up to about 5 hours, up to about 6 hours, up to about 7 hours, up to about 8 hours, up to about 9 hours, up to about 10 hours, up to about 12 hours, up to about 1 day, up to about 2 days, up to about 3 days, up to about 4 days, or up to about a week prior to DNA vector administration. In certain embodiments, the cytosolic DNA-sensing inhibitor is administered independently (with respect to the type 1 interferon receptor pathway inhibitor) at about the same time, up to about 5 minutes, up to about 15 minutes, up to about 30 minutes, up to about 45 minutes, up to about 60 minutes, up to about 90 minutes, up to about 2 hours, up to about 3 hours, or about 4 hours prior to DNA vector administration.

In certain embodiments the cytosolic DNA-sensing inhibitor is administered independently (with respect to the type 1 interferon receptor pathway inhibitor) about 15 minutes, about 30 minutes, about 45 minutes, about 60 minutes, about 90 minutes, about 120 minutes, or about 1 day after the DNA; at about the same time, about 5 minutes, about 10 minutes, about 15 minutes, about 30 minutes, about 45 minutes, about 60 minutes, about 90 minutes, about 2 hours, about 2.5 hours, about 3 hours, about 3.5 hours, or about 4 hours prior to DNA vector administration.

In certain embodiment, the DNA vector, the type 1 interferon receptor pathway inhibitor and the cytosolic DNA-sensing inhibitor, can be provided in the same nanoparticle to release at about the same time, or the nanoparticle can be designed to delay release of one of the components. In an embodiment, the nanoparticle is designed to release the type 1 interferon receptor pathway inhibitor and the cytosolic DNA-sensing inhibitor prior to the release of the DNA vector.

In certain embodiments additional immune inhibitors are not administered within 2 months prior to or within 2 months after administration of the type 1 interferon receptor inhibitor. Reference to “additional immune inhibitors” refers one or more (including any combination or all) inhibitor selected from a JAK inhibitor, a JAK1 or JAK2 inhibitor, a STAT inhibitor, a cGAS inhibitor, a STING inhibitor and/or an inflammasome pathway inhibitor. In further embodiments, the additional immune inhibitor is not administered within 1 day, 2 days, 3 days, 4 days, 5 days, 6 days, 7 days, 2 weeks, 1 month, or 2 months prior to type 1 interferon receptor inhibitor administration; and independently the additional immune inhibitor is not administered within 1 day, 2 days, 3 days, 4 days, 5 days, 6 days, 7 days, 2 weeks, 1 month, or 2 months after the type 1 interferon receptor inhibitor.

Reference to “independently” with respect to two different lists, such as times prior and times after, indicates each member of one list can be combined with each member of the other list. For example, no additional immune inhibitor is administered within 1 day prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 2 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 3 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 4 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 5 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 6 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 7 days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 2 weeks prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; no additional immune inhibitor administered within 1 months prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration; and no additional immune inhibitor administered within 2 months days prior to type 1 interferon receptor inhibitor administration can be combined with no additional immune inhibitor administration within 1 day, 2 days, 3 days, 4 days, 5 days, 6 days, 7 days, 2 weeks, 1 month, or 2 months after type 1 interferon receptor inhibitor administration.

Treatment doses of DNA vector can vary and depend upon the type, onset, progression, severity, frequency, duration, or probability of the disease or disorder to which treatment is directed, the clinical endpoint desired, previous or simultaneous treatments, the general health, age, gender, race or immunological competency of the subject and other factors that will be appreciated by the skilled artisan. The dose amount, number, frequency, or duration can be proportionally increased or reduced, as indicated by any adverse side effects, complications or other risk factors of the treatment or therapy and the status of the subject.

The dose to achieve a therapeutic effect, e.g., vector DNA dose in mg per kilogram of body weight (mg/kg), will also vary based on several factors including route of administration, the level of transgene expression required to achieve a therapeutic effect, the specific disease or disorder treated, host immune response to DNA, host immune response to transgene expression product, and the stability of the protein, peptide, or nucleic acid expressed. Based on the guidance provided herein, one skilled in the art can determine a suitable vector DNA dose range to treat a patient having a particular disease or disorder.

The overall level of transgene expression can vary depending upon the use of the DNA vector and the targeted disease or disorder. In different embodiments of gene therapy providing a therapeutic protein, the provided expression or activity is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the corresponding subject protein.

In certain embodiments, a method according to the instant invention can result in reduction of expression or activity of a protein targeted by a therapeutic nucleic acid. In different embodiments reduction of expression or activity of a protein targeted by a therapeutic nucleic acid is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the target protein.

Methods and uses of the instant invention include delivery and administration systemically, regionally or locally, for example, by injection or infusion. Delivery of the compositions in vivo can generally be accomplished, for example, by injection using a conventional syringe, although other delivery methods such as convection-enhanced delivery are envisioned (see, e.g., U.S. Pat. No. 5,720,720). For example, compositions can be delivered subcutaneously, epidermally, intradermally, intrathecally, intraorbitally, intramucosally, intraperitoneally (IP), intravenously (IV), intra-pleurally, intraarterially, orally, intrahepatically, via the portal vein, or intramuscularly. Other modes of administration include oral and pulmonary administration, suppositories, and transdermal applications.

### VII.A. Illustrative Disease and Disorder

Diseases and disorders that can be treated by vector DNA include lung disease (e.g., cystic fibrosis), a blood disorder (e.g., anemia), CNS diseases and disorder, epilepsy, a lysosomal storage disease (e.g., aspartylglucosaminuria), Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay-Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), and a disease of solid organs (e.g., brain, liver, kidney, heart).

Glycogen storage disease type II, also called Pompe disease, is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in body tissues, resulting in cardiac, respiratory, and skeletal muscle dysfunction.

Blood clotting disorders which can be treated include hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Other diseases and disorders that can be treated include bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

Other diseases and disorders that can be treated include proliferative diseases (e.g., cancers, tumors and dysplasias), Crigler-Najjar and metabolic diseases like metabolic diseases of the liver; Friedreich ataxia; infectious diseases; viral diseases induced for example by hepatitis B or C viruses, HIV, herpes, and retroviruses; genetic diseases such as cystic fibrosis, dystroglycanopathies, myopathies such as Duchenne muscular myopathy or dystrophy, myotubular myopathy, sickle-cell anemia, sickle cell disease, Fanconi's anemia, diabetes, amyotrophic lateral sclerosis (ALS), myotubularin myopathy, motor neuron diseases such as spinal muscular atrophy (SMA), spinobulbar muscular atrophy, or Charcot-Marie-Tooth disease; arthritis; severe combined immunodeficiencies such as RS-SCID, ADA-SCID or X-SCID; Wiskott-Aldrich syndrome; X-linked thrombocytopenia; X-linked congenital neutropenia; chronic granulomatous disease; clotting factor deficiencies; cardiovascular disease such as restenosis, ischemia, dyslipidemia, and homozygous familial hypercholesterolemia; eye or ocular diseases such as retinitis pigmentosa, X-linked retinitis pigmentosa, autosomal dominant retinitis pigmentosa, recessive retinitis pigmentosa, choroideremia, choroidal neovascularization, gyrate atrophy, retinoschisis, X-linked retinoschisis, macular degeneration, diabetic macular edema (DME), diabetic retinopathy associated with DME, wet age-related macular degeneration (wet AMD or wAMD), macular edema following retinal vein occlusion, non-arteritic ischaemic optic neuropathy, Leber congenital amaurosis, Leber hereditary optic neuropathy, achromatopsia, and Stargardt disease; lysosomal storage diseases such as San Filippo syndrome; hyperbilirubinemia such as CN type I or II or Gilbert's syndrome; glycogen storage disease such as GSDI, GSDII (Pompe disease), GSDIII, GSDIV, GSDV, GSDVI, GSDVII, GSDVIII or lethal congenital glycogen storage disease of the heart.

In certain embodiments, the subject has a disease or disorder that affects or originates in the central nervous system (CNS). In certain embodiments, the disease is a neurodegenerative disease. Non-limiting examples of CNS or neurodegenerative disease include Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the disease is a psychiatric disease, an addiction (e.g., to tobacco, alcohol, or drugs), epilepsy, Canavan's disease, or adrenoleukodystrophy. In certain embodiments, the CNS or neurodegenerative disease is a polyglutamine repeat disease such as, spinocerebellar ataxia (SCA1, SCA2, SCA3, SCA6, SCA7, or SCA17).

### VII.B. Administration Examples of Different Diseases and Conditions

Based on the present application a wide variety of different diseases and disorders can treated. Administration for certain disease or disorders is highlighted in the present section. The provided examples, like the other examples in the present application, are for providing different embodiments and illustration purposes.

For Pompe disease, an effective amount is an amount of GAA that inhibits or reduces glycogen production or accumulation, enhances or increases glycogen degradation or removal, reduces lysosomal alterations in tissues of the body of a subject, or improves muscle tone and/or muscle strength and/or respiratory function in a subject. Effective amounts can be determined, for example, by ascertaining the kinetics of GAA uptake by myoblasts from plasma. Myoblasts GAA uptake rates (K uptake) of about 141-147 nM appear to be effective (e.g., Maga et al., J. Biol. Chem. 2012, 8; 288(3), 1428). In animal models, GAA activity levels in plasma greater than about 1,000 nmol/hr/mL, for example, about 1,000 to about 2,000 nmol/hr/mL have been observed to be therapeutically effective.

For HemA and HemB it is generally expected that a blood coagulation factor concentration greater than 1% of factor concentration found in a normal individual is needed to change a severe disease phenotype to a moderate one. A severe phenotype is characterized by joint damage and life-threatening bleeds. To convert a moderate disease phenotype into a mild one, it is expected that a blood coagulation factor concentration greater than 5% of normal is needed.

FVIII normal level is about 100-200 ng/ml and FIX levels in normal humans is 5000 ng/ml, but levels can be more or less and still considered normal, due to functional clotting as determined, for example, by an activated partial thromboplastin time (aPTT) one-stage clotting assay. Thus, a therapeutic effect can be achieved such that the total amount of FVIII or FIX in the subject/human is greater than 1% of the FVIII or FIX present in normal subjects/humans, e.g., 1% of 100-300 ng/mL.

### VII.C. Combination Treatments

The DNA vectors described herein can be used in combination with other therapies for a particular disease or disorder.

### VIII. Kits

Further provided herein is a kit providing in separate containers at least: (a) a pharmaceutical composition comprising a nanoparticle comprising a DNA; (b) a type 1 interferon receptor pathway inhibitor; (c) optionally a cGAS-STING pathway inhibitor; (d) optionally an inflammasome pathway inhibitor; and (e) optionally an immune cell modulator. The amounts of different components can be obtained taking into account different factors such as the targeted disease or disorder, and the DNA vector. The kit may also provide a label with instructions for administration according to the methods described herein.

### IX. Additional Aspects and Embodiments

Additional aspects, embodiments, and combinations thereof include the following:

A first aspect of the invention describes a method of intracellular delivery of a DNA to a subject comprising the steps of administering:
a) a type 1 interferon receptor pathway inhibitor; and
b) a first nanoparticle comprising the DNA; wherein step (b) is performed prior to, concomitantly with, or after step (a).

Embodiment E1 further describes first aspect wherein the DNA is a DNA vector comprising a transgene operatively linked to a regulatory element. In further embodiments the transgene is operatively linked to a promoter; is operatively linked to a promoter/enhancer; is operatively linked to promoter/enhancer, a polyadenylation signal sequence, and/or a regulatable element; and the DNA vector comprises 5′ to 3′ the promoter/enhancer, the transgene, and the polyadenylation signal sequence.

Reference to 5′ to 3′ with respect to specified elements indicates the relative position of different elements, does not require the different elements be adjacent to each other, and allows for the presence of additional sequences. The additional sequences, which in some cases, provides for additional activity, can be located in different positions such as between two identified elements, at the ‘3 end, or at the 5’ end.

Embodiment E2 further describes the first aspect and E1 wherein the type 1 interferon receptor pathway inhibitor is a type 1 interferon receptor inhibitor. In different embodiments, the receptor inhibitor is an antibody that binds to the type 1 interferon receptor, or comprises an antibody fragment that binds to the type 1 interferon receptor. Antibody binding fragments contain three complementary determining regions in a variable region framework allowing for antigen binding. In a further embodiment, the antibody is anifrolumab.

Reference to a particular embodiment includes reference to further and different embodiments provided therein. For example, reference in the second embodiment to the first embodiment provides a reference to all the embodiments provided in the first embodiment including the further and different embodiments provided therein.

Embodiment E3 further describes the first aspect and E1 wherein the type 1 interferon receptor pathway inhibitor is a Janus activated kinase inhibitor. In different embodiments, the Janus activated kinase inhibitor is Janus activated kinase 1 inhibitor or a tyrosine kinase 2 inhibitor; is a selective Janus activated kinase 1 inhibitor; is a selective tyrosine kinase 2 inhibitor; or is a compound of Table 1 or a pharmaceutically acceptable salt thereof.

Embodiment E4 further describes the first aspect and E1 wherein the type 1 interferon receptor pathway inhibitor is a signal transducer and activator of transcription (STAT) protein inhibitor. In different embodiments, the STAT inhibitor is a STAT1 inhibitor or STAT2 inhibitor; or is a compound of Table 2, or a pharmaceutically acceptable salt thereof.

Embodiment E5 further describes the first aspect, E1, E2, E3 and E4 wherein in different embodiments, the method inhibits cytokines production; or inhibits IFN gamma, IFN alpha, and/or of IL-6 production.

Embodiment E6 further describes the first aspect, E1, E2, E3, E4, and E5, wherein the method further comprises administration of a cGAS-STING pathway inhibitor. In further embodiments the cGAS-STING pathway inhibitor is a compound of any of Tables 3, 4, or 5, or a pharmaceutically acceptable salt thereof.

Embodiment E7 further describes the first aspect, E1, E2, E3, E4, E5, and E6, wherein the method further comprises administration of an inflammasome pathway inhibitor. In further embodiments the inflammasome pathway inhibitor is a polynucleotide having the sequence of SEQ ID NO: 1 or SEQ ID NO: 2.

Embodiment E8 further describes the first aspect, E1 and E2, wherein a JAK inhibitor is not administered within 2 months prior to or 2 months after type 1 interferon receptor inhibitor administration; a JAK1 or JAK2 inhibitor is not administered within 2 months prior to or 2 months after type 1 interferon receptor inhibitor administration; a STAT inhibitor is not administered within 2 months prior to or 2 months after type 1 interferon receptor inhibitor administration; a cGAS inhibitor is not administered within 2 months prior to or 2 months after type 1 interferon receptor inhibitor administration; a STING inhibitor is not administered within 2 months prior to or 2 months after interferon receptor inhibitor administration; and/or a inflammasome pathway inhibitor is not administered within 2 months prior to or 2 months after type 1 interferon receptor inhibitor; a JAK inhibitor, a STAT inhibitor, a cGAS inhibitor, a STING inhibitor and a inflammasome pathway inhibitor is not administered within 2 months prior to or 2 months after type 1 interferon receptor inhibitor administration. In further embodiments, the inhibitor provided in E7 (other than the type 1 interferon receptor inhibitor) is not administered within 1 day, 2 days, 3 days, 4 days, 5 days, 6 days, 7 days, 2 weeks, 1 month, or 2 months prior to type 1 interferon receptor inhibitor administration; and independently within 2 days, 3 days, 4 days, 5 days, 6 days, 7 days, 2 weeks, 1 month, or 2 months after the type 1 interferon receptor inhibitor.

Embodiment E9 further describes the first aspect, E2, E3, E4, E5, E6, E7, and E8 wherein the DNA vector comprises a transgene encoding a viral antigen, a bacterial antigen, a therapeutic protein, a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN). In further embodiments the therapeutic protein is as provided in Section III.C. supra.

Embodiment E10 further describes the first aspect, E1, E2, E3, E4, E5, E6, E7, E8, and E9 wherein the type 1 interferon receptor pathway inhibitor is provided in a second nanoparticle. In further embodiments, the second nanoparticle has substantially the same composition as the first nanoparticle; and the first and second nanoparticles are lipid nanoparticles or lipid polymer nanoparticles. In a further embodiment, the nanoparticle is an LNP; and the nanoparticle is as described in Section I.B. supra.

Embodiment E11 further describes the first aspect, E1, E2, E3, E4, E5, E6, E7, E8, and E9, wherein the type 1 interferon receptor pathway inhibitor is provided along with the DNA or DNA vector in the first nanoparticle. In further embodiments the first nanoparticle is a lipid nanoparticle; the first nanoparticle is a lipid polymer nanoparticle; the first nanoparticle is an exosome; the first nanoparticle is configured to release the inhibitor prior to the release of the DNA or DNA vector; the first nanoparticle is lipid nanoparticle configured to release the inhibitor prior to the release of the DNA or DNA vector; the first nanoparticle is a lipid polymer nanoparticle configured to release the inhibitor prior to the release of the DNA or DNA vector; and the first nanoparticle is a exosome configured to release the inhibitor prior to the release of the DNA or DNA vector.

Embodiment E12 further describes the first aspect, E1, E2, E3, E4, E5, E6, E7, E8, E9, and E10 wherein the type 1 interferon receptor pathway inhibitor is administered at about the same time, prior to or after administration of the DNA or DNA vector. In different embodiments, the type 1 interferon receptor pathway inhibitor is administered at least 30 minutes, at least 60 minutes, at least 90 minutes, or at least 120 minutes prior to the DNA or DNA vector; the type 1 interferon receptor pathway inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, up to about 4 hours, up to about 5 hours, up to about 6 hours, up to about 7 hours, up to about 8 hours, up to about 9 hours, up to about 10 hours, up to about 12 hours, up to about 1 day, up to about 2 days, up to about 3 days, up to about 4 days, or up to about a week prior to DNA or DNA vector. In additional embodiments, the type 1 interferon receptor pathway inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, or up to about 4 hours prior to the DNA or DNA vector.

Embodiment E13 further describes the first aspect, E1, E2, E3, E4, E5, E6, E7, E8, E9, E10, E11 and E12, wherein two or more different type 1 interferon receptor pathway inhibitors are used. The different inhibitors can be provided without a nanoparticle, can be provided in different nanoparticles, or can be provided in the same nanoparticle.

Embodiment E14 further describes the first aspect, E1, E2, E3, E4, E5, E6, E7, E8, E9 E10, E11, E12 and E13, wherein the DNA and DNA vector either substantially comprise double-stranded DNA or the DNA and DNA vector substantially comprise single-stranded DNA. In different embodiments, more than half, at least 75%, at least 90%, at least 95%, or at least 99% of the DNA or DNA vector is double-stranded DNA or 100% of the DNA is double-stranded.

Embodiment E15 further describes first the aspect, E1, E2, E3, E4, E5, E6, E7, E8, E9, E10, E11, E12, E13 and E14 wherein the subject is a human patient. In a further embodiment, the method provides a therapeutically effective amount of a transgene.

A second aspect of the present invention features a nanoparticle comprising:
a) a DNA; and
b) a type 1 interferon receptor pathway inhibitor.

Embodiment E16 further describes the second aspect, wherein the DNA is a DNA vector comprising a transgene, and the transgene is operatively linked to a regulatory element. In further embodiments, the transgene is operatively linked to a promoter; is operatively linked to a promoter enhancer; is operatively linked to promoter/enhancer, a polyadenylation signal sequence and/or regulatable element; and the DNA vector comprises 5′ to 3′ the promoter/enhancer, the transgene, and the polyadenylation signal sequence.

Embodiment E17 further describes the second aspect and E16, wherein the type 1 interferon receptor pathway inhibitor is not a type 1 interferon receptor inhibitor.

Embodiment E18 further describes the second aspect and E16 wherein the type 1 interferon receptor pathway inhibitor is a Janus activated kinase inhibitor. In different embodiments, the Janus activated kinase inhibitor is Janus activated kinase inhibitor 1 or a tyrosine kinase 2 inhibitor; or is a compound of Table 1, or a pharmaceutically acceptable salt thereof.

Embodiment E19 further describes the second aspect and E16 wherein the type 1 interferon receptor pathway inhibitor is a signal transducer and activator of transcription (STAT) protein inhibitor. In different embodiments, the STAT inhibitor is a STAT1 inhibitor or a STAT2 inhibitor; or is a compound of Table 2, or a pharmaceutically acceptable salt thereof.

Embodiment E20 further describes the second aspect, E16, E17, E18, and E19 wherein the composition further comprises a cGAS-STING pathway inhibitor. In further embodiments the cGAS-STING pathway inhibitor is a compound of any of Tables 3, 4, or 5, or a pharmaceutically acceptable salt thereof.

Embodiment E21 further describes the second aspect, E16, E17, E18, E19, and E20, wherein the composition further comprises an inflammasome pathway inhibitor. In further embodiments the inflammasome pathway inhibitor is a polynucleotide having the sequence of SEQ ID NO: 1 or SEQ ID NO: 21.

Embodiment E22 further describes the second aspect, E16, E17, E19, E20 and E21, wherein the DNA is a DNA vector comprising a transgene encoding a viral antigen, a bacterial antigen, a therapeutic protein, a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN). In further embodiments the therapeutic protein is as provided in Section III. C. supra.

Embodiment E23 further describes the second aspect, E16, E17, E18, E19, E20, E21 and E22, wherein the nanoparticle is a lipid nanoparticle, a lipid polymer nanoparticle, or an exosome. In further embodiments the nanoparticle is a lipid nanoparticle; the nanoparticle is a lipid polymer nanoparticle; the nanoparticle is an exosome; the nanoparticle is lipid nanoparticle configured to release the inhibitor prior to the release of the DNA or DNA vector; the nanoparticle is a lipid polymer nanoparticle configured to release the inhibitor prior to the release of the DNA or DNA vector; and the nanoparticle is an exosome configured to release the inhibitor prior to the release of the DNA or DNA vector. In further embodiments, the nanoparticle is an LNP, such as those described in Section I.B. supra.

E24 further describes the second aspect, E16, E17, E18, E19, E20, E21, E22, and E23, wherein the DNA and DNA vector each substantially comprise double-stranded DNA or the DNA and DNA vector each substantially comprise single-stranded DNA. In different embodiments, more than half, at least 75%, at least 90%, at least 95%, or at least 99% of the DNA or DNA vector is double-stranded DNA or 100% of the DNA is double-stranded.

A third aspect is directed to a pharmaceutical composition comprising the nanoparticle composition of the second aspect, E16, E17, E18, E19, E20, E21, E22, E23 and 24, and a pharmaceutically acceptable carrier.

A fourth aspect is directed to a pharmaceutical composition for use in medicine (preferably gene therapy) comprising a DNA vector comprising a transgene, wherein the composition is for use prior to, concomitantly with, or after administration of a type 1 interferon receptor pathway inhibitor, wherein the DNA vector is provided in a nanoparticle. Additional embodiments are provided in the methods and components described in the first aspects and related embodiments; the compositions described in the second aspect and related embodiments; and the pharmaceutical composition of the third aspect.

A fifth aspect of the invention is directed to a method of making a medicament for medicine (preferably gene therapy) comprising a nanoparticle comprising a DNA vector wherein the medicament is for use prior to, concomitantly with, or after administration of a type 1 interferon receptor pathway inhibitor, wherein the DNA vector and/or type 1 interferon receptor pathway inhibitor is combined with a pharmaceutical acceptable carrier, where the nanoparticle and DNA vector is as described in the first aspect and related embodiments and the second aspect and related embodiments.

A sixth aspect is directed to a compound having the structure

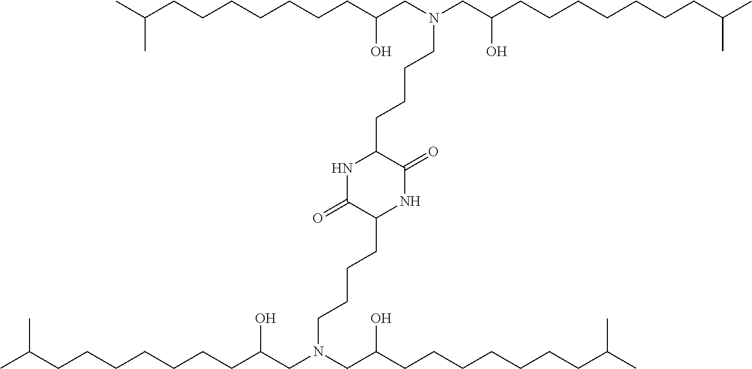 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

or a pharmaceutically acceptable salt thereof. In further embodiments, the compound is present in an LNP as provided in any of first to fifth aspects, or accompany embodiments (E1-E23).

A number of different aspect and embodiments of the instant invention have been described throughout the application. Nevertheless, the skilled artisan without departing from the spirit and scope of the instant invention, can make various changes and modifications of the instant invention to adapt it to various usages and conditions.

### EXAMPLES

Examples are provided below further illustrating different features of the present invention and methodology for practicing the invention. The provided examples do not limit the claimed invention.

### Example 1: Ablation of Interferon Receptor Signaling

Survival and expression studies were performed with wild type (WT) and IFNAR-deficient knock-out (IFNAR KO) mice (n=5 per group) dosed systemically at t=0, week 7, and week 12 with 1.25 mpk (25 μg) of human factor IX (hFIX) transgene-encoding plasmid DNA encapsulated in lipid nanoparticles (DNA-LNP). The LNP used in the study was formulated with cKK-E12.

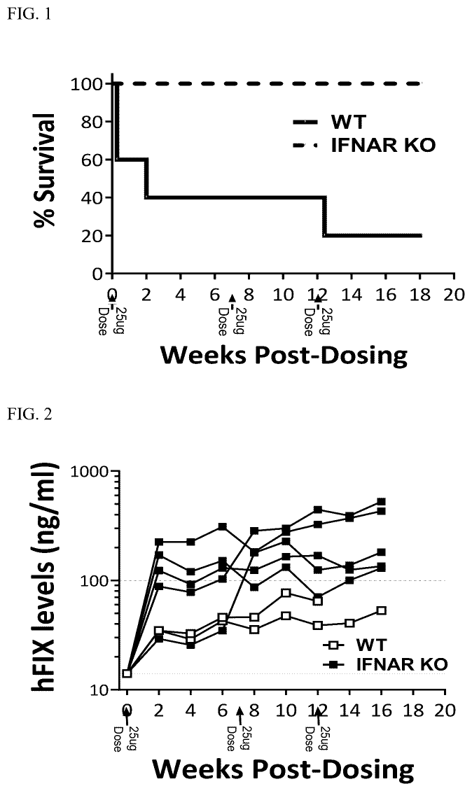

Ablation of IFNAR signaling improved DNA-LNP tolerability and transgene expression. FIG. 1 illustrates survival of WT and IFNAR KO out to 18 weeks. FIG. 2 illustrates transgenic hFIX protein in blood plasma measured by ELISA at various points post-dosing in surviving mice from each group out to 16 weeks. Therefore, IFNAR signaling could be an impediment to tolerability and efficacy of DNA-LNP gene therapies.

### Example 2: Inhibiting Interferon Receptor Signaling

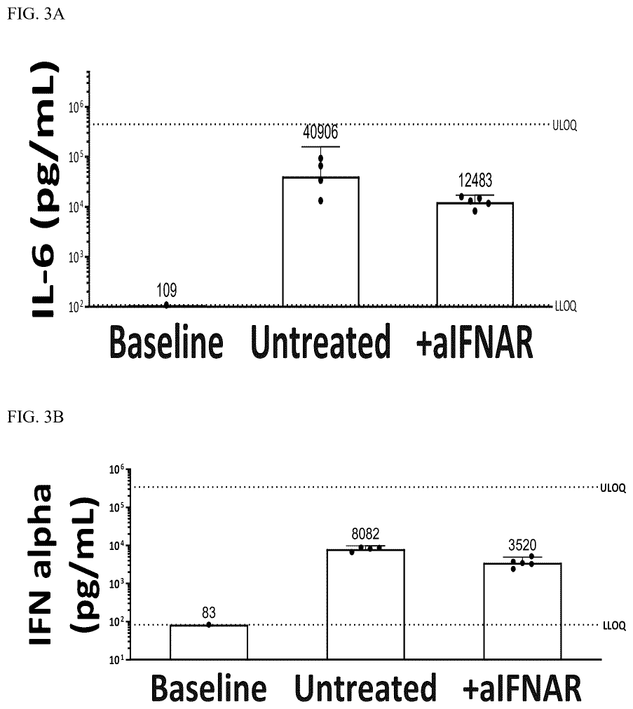

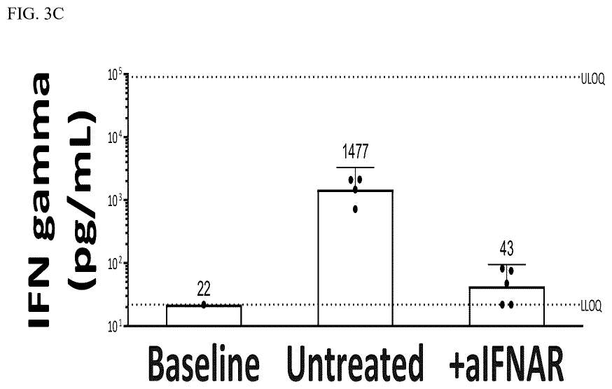

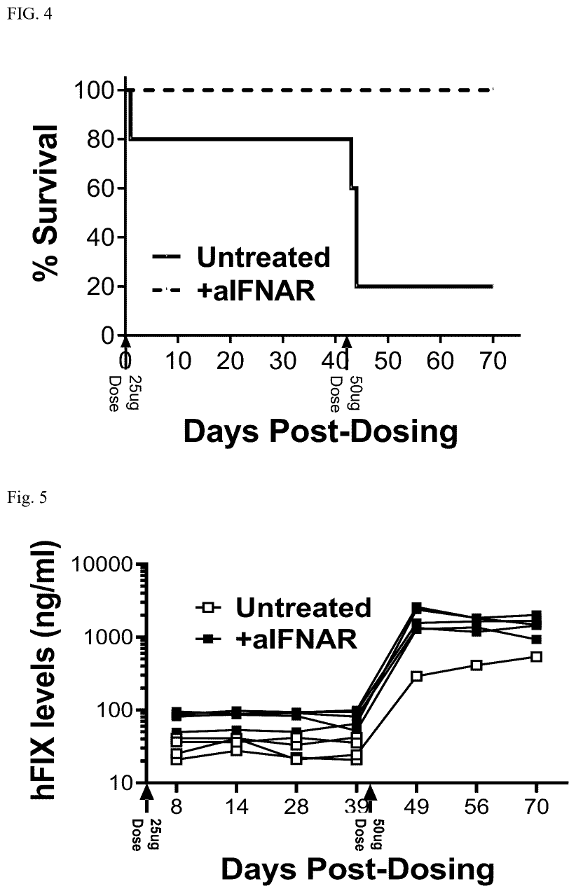

Plasma cytokines levels, % survival, and expression levels were measured using WT mice that were untreated or treated (n=5 per group) once with 15 mpk anti-mouse IFNAR blocking antibody (aIFNAR). The employed aIFNAR was MAR1-5A3. (Dunn et al., Nat Immunol. 2005 July; 6(7):722-9). Blocking antibody was added 3 hours prior to systemically dosing at t=0 with 1.25 mpk (25 μg) of hFIX DNA-LNP and again at day 41 with 2.5 mpk (50 μg) of hFIX DNA-LNP. Plasma cytokine levels were assayed at 4 hours post day 41 dosing and compared to pooled plasma pre-dosing levels (“Baseline”). The LNP used in the study was formulated with bCKK-E12. FIG. 3A illustrates IL-6 levels, FIG. 3B illustrates IFN alpha levels and FIG. 3C illustrates IFN gamma levels. Survival and hFIX expression was followed out to 70 days. FIG. 4 illustrates survival out to day 70. FIG. 5 illustrates transgenic hFIX protein in blood plasma measured by ELISA at various points post-dosing in surviving mice from each group out to day 70.

These data show that antibody treatment-mediated inhibition of IFNAR signaling reduces acute inflammatory responses and improves survival of mice dosed with a DNA-LNP gene therapy. Anti-IFNAR treatment improved DNA-LNP transgene efficacy compared to mice not treated with anti-IFNAR. Blocking the IFNAR pathway could have the dual benefit of improving both tolerability and efficacy of DNA-LNP-delivered gene therapies.

### Example 3: Targeting JAK1 and JAK2 Kinases Downstream of IFNAR

Wild type mice were untreated, or treated (n=5 per group) with JAK inhibitors ruxolitinib or baricitinib orally 30 minutes prior to, and days 1, 2, and 3 post systemic dosing with 2.5 mpk (50 μg) of human factor IX (hFIX) transgene-encoding plasmid DNA encapsulated in lipid nanoparticles (DNA-LNP). The LNP used in the study was formulated with bCKK-E12. Survival of mice in each group was followed out to 30 days (FIG. 6).

Ruxolitinib and or baricitinib, which are selective for JAK1 and JAK2, dramatically improved survival of mice dosed with a DNA-LNP gene therapy. JAK inhibitor treatment may act (at least in part) to improve DNA-LNP tolerability by blocking IFNAR signaling.

### Example 4: IFNAR Neutralization Enhances Transgene Expression

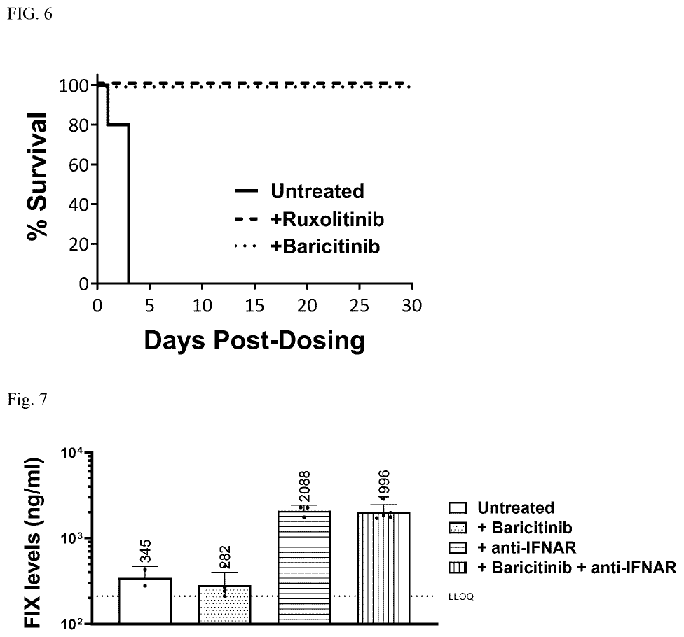

Wild type C57BL/6 mice were untreated, or treated (n=5 per group) once IP with 15 mpk anti-mouse IFNAR blocking antibody (“anti-IFNAR”; clone MAR1-5A3, described in Dunn et al., Nat Immunol. 2005 July; 6(7):722-9) 3 hours prior to systemically (IV tail injections) dosing with 2.5 mpk (50 μg) of human factor IX (hFIX) transgene-encoding plasmid DNA encapsulated in lipid nanoparticles (DNA-LNP). The LNP used in the study was formulated with bCKK-E12. Mice were treated or not treated with the oral JAK inhibitor baricitinib (“Bar”) 90 minutes prior to DNA-LNP dosing, and then daily on days 1-6 post-DNA-LNP dosing. Transgenic hFIX protein in blood plasma of surviving mice was measured by ELISA one-week post-DNA-LNP dosing (FIG. 7).

Anti-IFNAR boosted DNA-LNP transgene efficacy. Baricitinib (selective for JAK1 and JAK2) did not boost transgene efficacy and anti-IFNAR in combination with baricitinib did not boost transgene efficacy.

### Example 5: STING Ablation

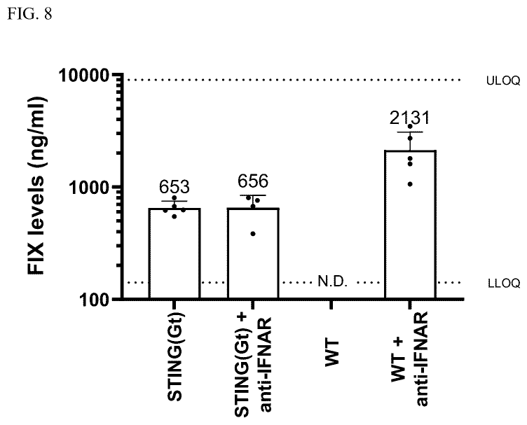

Wild type C57BL/6 (“WT”) mice and STING-deficient mice (i.e., mice harboring the Goldenticket nonsense mutation (“STING (Gt)”)) were untreated, or treated IP (n=5 per group) once with 15 mpk anti-mouse IFNAR blocking antibody (“anti-IFNAR”), 3 hours prior to systemic (IV tail injections) dosing with 5 mpk (100 mg) of human factor IX (hFIX) transgene-encoding plasmid DNA encapsulated in lipid nanoparticles (DNA-LNP). All WT mice not treated with anti-IFNAR died within two days post-DNA-LNP dosing. Transgenic hFIX protein in blood plasma of surviving mice was measured by ELISA four weeks post-DNA-LNP dosing (FIG. 8). The data suggest that complete ablation of STING signaling abrogates the positive effect that blocking IFNAR signaling has on transgene expression.

### Example 6: Effect of Anti-INFAR Antibody Delivered by Different LNPs

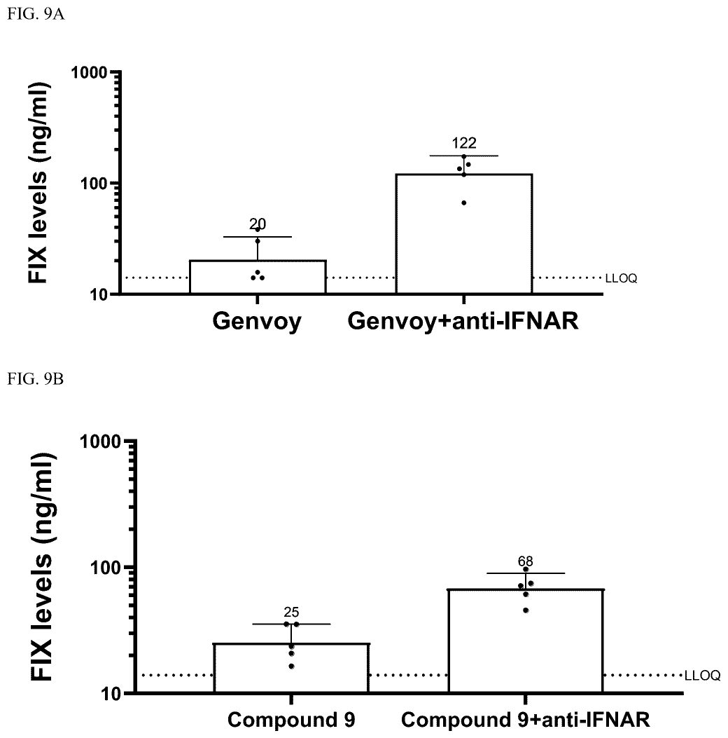

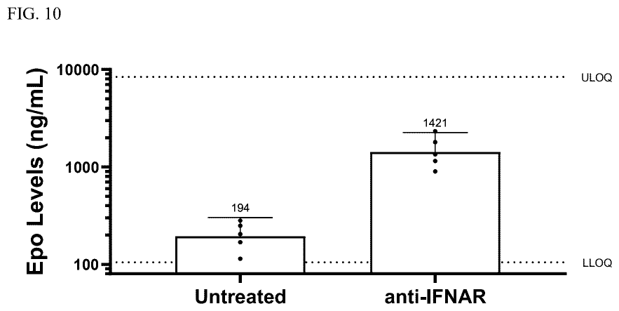

Wild type C57BL/6 mice were untreated or treated IP (n=5 per group) once with 15 mpk anti-mouse IFNAR blocking antibody (“anti-IFNAR”, as described in Example 2), 3 hours prior to systemic (IV tail injections) dosing with 2.5 mpk (50 mg) of human factor IX (“FIX”) transgene-encoding plasmid DNA. FIX transgene-encoding plasmid DNA encapsulated in lipid nanoparticles formulated using different lipid formulations and ionizable lipids. FIG. 9A illustrates results using Gen Voy-ILM™ and FIG. 9B illustrates results using Compound 9 lipid. According to Roces et al., Pharmaceutics, 2020, 12,1095, Gen Voy-ILM™ is made up of ionizable lipid, about 50%; DSPC, about 10%; cholesterol, about 37.5%; and stabilizer (PEG-Lipid), about 2.5%. Transgenic FIX protein in blood plasma was measured by ELISA one-week post-DNA-LNP dosing. LNPs comprising Compound 9 in the present examples were made of, approximately, Compound 9 (U.S. Patent Publication No. US2022204439) 50%; C14-PEG2000, 2.5%; cholesterol, 37.5%; and DSPC, 10%). The different LNP compositions comprising the anti-IFNAR antibody, increased transgene expression.

### Example 7: Effect of Anti-INFAR Antibody on EPO Expression in Babl/c Mice

Wild type Balb/c mice were untreated or treated IP (n=5 per group) once with 15 mpk anti-mouse IFNAR blocking antibody (“anti-IFNAR”, as described in Example 2), 3 hours prior to systemic (IV tail injections) dosing with 2.5 mpk (50 mg) of human erythropoietin (“EPO”) expression plasmid DNA encapsulated in lipid nanoparticles (DNA-LNP; the LNP used in the study was formulated with bCKK-E12) Transgenic EPO protein in blood plasma was measured by ELISA one week post-DNA-LNP dosing. The results are illustrated in FIG. 10. The anti-IFNAR treatment results in an increased on expression of DNA-LNP-delivered EPO transgene on Balb/c mice.

### Example 8: bCKK-E12 Synthesis

bCKK-E12 was synthesized using the following procedure:

### Step A:

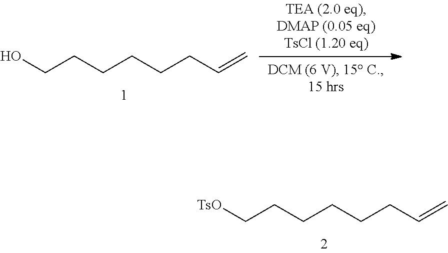

A 1000 mL three-necked round bottom flask was equipped with a magnetic stirrer, an addition funnel and a thermometer. To a solution of compound 1(50.0 g, 390 mmol, 1.0 eq) in dichloromethane (DCM) (300 mL) was added 4-toluenesulfonyl chloride (TsCl) (89.2 g, 468 mmol, 1.2 eq) and triethylamine (TEA) (78.9 g, 780 mmol, 2.0 eq) and DMAP (2.38 g, 19.5 mmol, 0.05 eq). The resulting mixture was stirred at 15° C. for 15 hours. Thin layer chromatography (TLC) (petroleum ethe/ethyl acetate=10/1, R_{f}=0.7) indicated that compound 1 was consumed completely and two new spots were formed. The reaction was clean according to the TLC. The reaction mixture was quenched by addition of H2O (600 mL) at 25° C., and then extracted with DCM (500 mL, 300 mL). The combined organic layers were concentrated under reduced pressure to give a residue. The residue was purified by column chromatography (SiO₂, petroleum ether/ethyl acetate=100/1 to 50/1) to afford compound 2(100 g, 354 mmol, 90.8% yield) as a yellow oil.

Compound 2: ¹H NMR: ET72382-2-PIB 400 MHZ, CDCl₃ δ 7.80 (d, J=8.4 Hz, 2H), 7.35 (d, J=8.0 Hz, 2H), 5.43-7.81 (m, 1H), 4.88-5.05 (m, 2H), 4.03 (t, J=6.4 Hz, 2H), 2.46 (s, 3H), 2.01 (q, J=7.2 Hz, 2H), 1.59-1.72 (m, 2H), 1.15-1.39 (m, 6H).

### Step B:

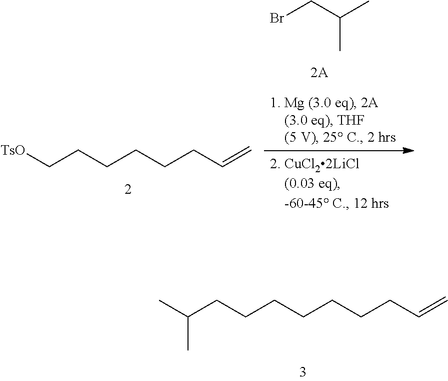

Step B: A 3000 mL three-necked round bottom flask was equipped with a magnetic stirrer, an addition funnel and a thermometer. A mixture of Mg (24.5 g, 1.01 mol, 3.0 eq) in tetrahydrofuran (THF) (400 mL) was added to the solution of compound 2A (138 g, 1.01 mol, 3.0 eq) in THF (100 mL) at 30° C., and the resulting reaction mixture was stirred at 45° C. for 2 hours. The reaction mixture was cooled to −60° C. and compound 2(95.0 g, 336 mmol, 1.0 eq) in THF (100 mL) and CuCl₂·2LiCl (0.1 M, 101 mL, 0.03 eq) was added. The mixture was stirred at 15° C. for 12 hours under N₂ atmosphere. TLC (petroleum ether: ethyl acetate=1:0, Rf=0.85) indicated compound 2 was completely consumed and one major new spot with lower polarity was detected. The reaction mixture was quenched by addition with saturated aq. NH₄Cl (1000 mL) and then extracted with petroleum ether (1000 mL, 700 mL). The combined organic layers were concentrated under reduced pressure to afford compound 3 (55.0 g, crude) as colorless oil, which was used for the next step without purification.

Compound 3: ¹H NMR: ET72382-4-PIB 400 MHZ, CDCl₃ δ 5.79-5.87 (m, 1H), 4.89-5.07 (m, 2H), 2.05 (q, J=6.8 Hz, 2H), 1.47-1.61 (m, 2H), 1.35-1.43 (m, 2H), 1.13-1.30 (m, 9H), 1.10-1.20 (m, 2H), 0.87 (d, J=6.8 Hz, 8H).

### Step C:

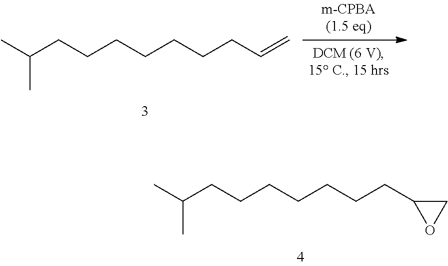

A 1000 mL three-necked round bottom flask was equipped with a magnetic stirrer, an addition funnel and a thermometer. To a solution of compound 3 (58.0 g, 345 mmol, 1.0 eq) in DCM (350 mL) was added m-CPBA (meta-chloroperoxybenzoic acid) (112 g, 517 mmol, 80% purity, 1.5 eq) at 15° C. The resulting mixture was stirred at 15° C. for 15 hours. TLC (petroleum ether/ethyl acetate=5/1, R_{f}=0.6) indicated compound 3 was completely consumed and one major new spot with larger polarity was detected. The mixture was poured into NaHSO₃ (1000 mL) and extracted with DCM (600 mL, 400 mL), and the organic layers were combined and washed with NaHCO₃ (600 mL) and brine (100 mL). The combined organic layers were concentrated under reduced pressure to give a residue. The residue was purified by column chromatography (SiO₂, petroleum ether/ethyl acetate=100/1 to 10/1) to afford compound 4 (37.0 g, 201 mmol, 58.3% yield) as a colorless oil.

Compound 4: ¹H NMR: ET72382-5-P1B 400 MHZ, CDCl₃ δ 2.87-2.98 (m, 1H), 2.75 (t, J=4.8 Hz, 1H), 2.47 (dd, J=4.8, 2.8 Hz, 1H), 1.40-1.61 (m, 5H), 1.22-1.39 (m, 7H), 1.22-1.39 (m, 1H), 1.08-1.19 (m, 2H), 0.87 (d, J=6.8 Hz, 6H).

### Step D:

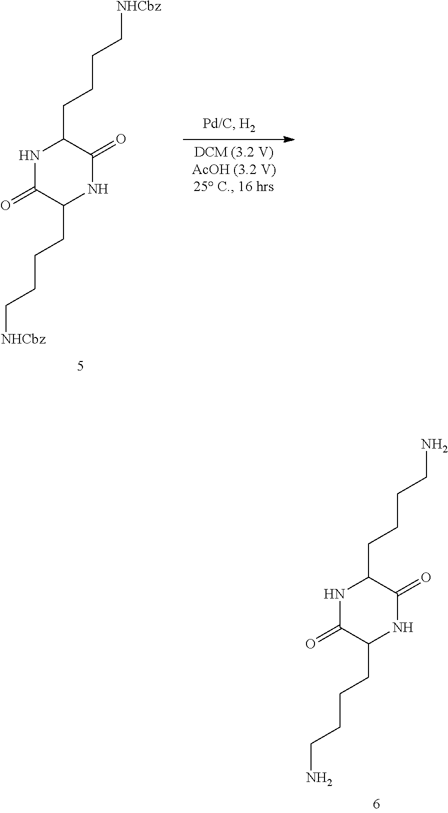 [SMILES: O=C(NCCCCC1NC(=O)C(CCCCNC(=O)OCc2ccccc2)NC1=O)OCc1ccccc1 ; NCCCCC1NC(=O)C(CCCCN)NC1=O]

To a solution of compound 5 (25.0 g, 47.6 mmol, 1.0 eq) in DCM (80 mL) and AcOH (80 mL) was added Pd/C (5.0 g, 10% purity) under N₂. The resulting suspension was degassed under vacuum and purged with H₂ several times. The mixture was stirred under H₂ (50 psi) at 25° C. for 16 hours. TLC (dichloromethane/methanol=10/1, R_{f}=0.1) indicated compound 5 was completely consumed and one new spot was formed. The reaction was clean according to TLC. The mixture was filtered and the cake was washed with MeOH (1000 mL). The filtrate was concentrated to give a residue. The residue was purified by re-crystallization from ethyl acetate (EtOAc) (350 mL) at 25° C. The solid was filtered and washed with EtOAc (100 mL) and dried under vacuum. The dried solid was dissolved in MeOH (150 mL) and the pH value of the mixture was adjusted to 8 with alkaline resin for twice. Compound 6(8.00 g, 31.2 mmol, 65.5% yield) was obtained as a white solid.

Compound 6: ¹H NMR: ET72382-1-P1B 400 MHZ, MeOD δ 3.94-4.07 (m, 2H), 2.60-2.71 (m, 4H), 1.73-1.94 (m, 4H), 1.31-1.57 (m, 8H).

### Step E:

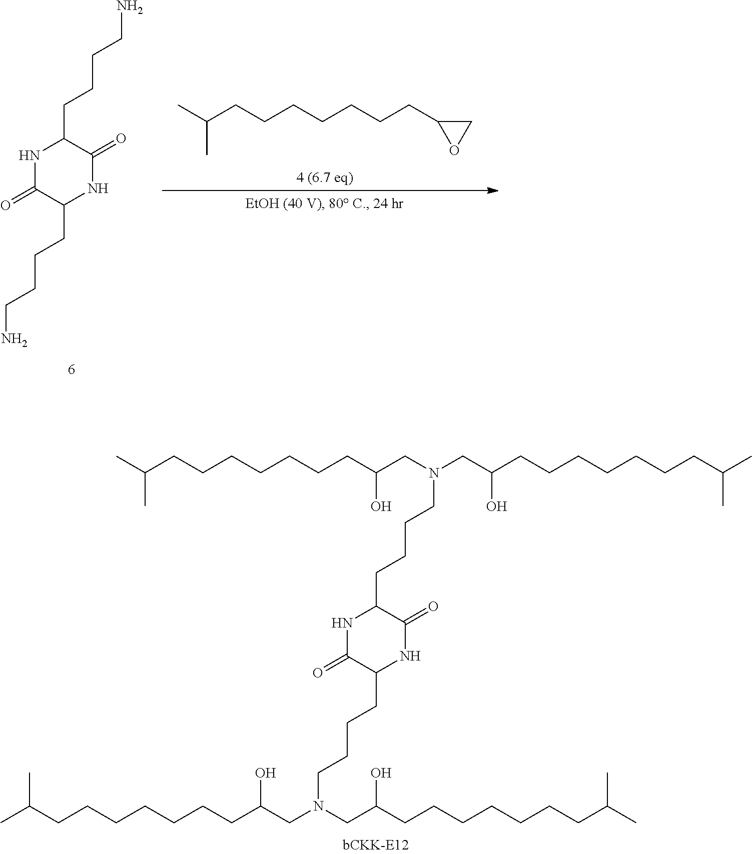 [SMILES: NCCCCC1NC(=O)C(CCCCN)NC1=O ; CC(C)CCCCCCCC1CO1 ; CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

A mixture of compound 6 (7.00 g, 27.3 mmol, 1.0 eq) and compound 4 (33.6 g, 182 mmol, 6.7 eq) in EtOH (280 mL) was degassed and purged with N2 3 times, and the resulting mixture was stirred at 80° C. for 24 hours under N₂ atmosphere. LC-MS showed compound 6 was consumed completely and one main peak with the desired mass was detected. The mixture was concentrated to give a residue. The residue was purified by column chromatography (SiO₂, DCM: MeOH=20/1 to 5/1) (TLC: DCM/MeOH=10/1, R_{f}=0.45) to afford compound bCKK-E12(10.5 g, 10.6 mmol, 38.7% yield) as a yellow gum.

Compound bCKK-E12: ¹H NMR: ET72382-7-P1B 400 MHZ, MeOD δ 3.93-4.05 (m, 2H), 3.63 (br s, 4H), 2.31-2.66 (m, 12H), 1.74-1.93 (m, 4H), 1.28-1.58 (m, 60H), 1.14-1.23 (m, 8H), 0.88 (d, J=6.8 Hz, 24H).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

I/We claim:

1. A method of intracellular delivery of a DNA to a subject comprising administration of:
a) a type 1 interferon receptor pathway inhibitor; and
b) a first nanoparticle comprising said DNA wherein step (b) can be performed prior to, concomitantly with, or after step (a).

2. The method of claim 1, wherein said DNA is a DNA vector comprising a transgene operatively linked to a regulatory element.

3. The method of claim 2, wherein said transgene is operatively linked to a promoter; and said DNA vector comprises 5′ to 3′ said promoter, said transgene, and a polyadenylation signal sequence.

4. The method of any one of claims 1-3, wherein said type 1 interferon receptor pathway inhibitor is a type 1 interferon receptor inhibitor.

5. The method of claim 4, wherein said type 1 interferon receptor inhibitor is an antibody that binds to the type 1 interferon receptor, or comprises an antibody fragment that binds to the type 1 interferon receptor.

6. The method of claim 5, wherein said antibody is anifrolumab.

7. The method of any one of claims 1-3, wherein said type 1 interferon receptor pathway inhibitor is a Janus activated kinase 1 inhibitor or a tyrosine kinase 2 inhibitor.

8. The method of claim 7, wherein said type 1 interferon receptor pathway inhibitor is a compound of Table 1 or a pharmaceutically acceptable salt thereof.

9. The method of any one of claims 1-3, wherein said type 1 interferon receptor pathway inhibitor is a signal transducer and activator of transcription (STAT) protein inhibitor.

10. The method of claim 9, wherein said STAT inhibitor is a STAT1 inhibitor or STAT2 inhibitor.

11. The method of claim 9, wherein said STAT inhibitor is a compound of Table 2 or a pharmaceutically acceptable salt thereof.

12. The method of any one of claims 1-11, wherein said method inhibits IFN gamma induced by DNA administration.

13. The method of any one of claims 1-12, further comprising the administration of a cyclic GMP-AMP synthase-stimulator of interferon genes (cGAS-STING) pathway inhibitor.

14. The method of claim 13, wherein said cGAS-STING pathway inhibitor is a compound of any of Tables 3, 4, or 5, or a pharmaceutically acceptable salt thereof.

15. The method of any of claims 1-14, further comprising the administration of an inflammasome pathway inhibitor.

16. The method of claim 14, wherein said inflammasome pathway inhibitor is a polynucleotide having the sequence of SEQ ID NO: 1 or SEQ ID NO: 2.

17. The method of any one of claims 4-6, wherein a JAK inhibitor, a STAT inhibitor, a cGAS inhibitor, a STING inhibitor and/or an inflammasome pathway inhibitor are not administered within 2 months prior to or 2 months after said type 1 interferon receptor inhibitor.

18. The method of any one of claims 2-16, wherein said transgene encodes a viral antigen, a bacterial antigen, a therapeutic protein, a short hair pin RNA (shRNA), a small interfering RNA (siRNA), a microRNA (miRNA), an RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN).

19. The method of any one of claims 1-18, wherein said type 1 interferon receptor pathway inhibitor is provided in a second nanoparticle.

20. The method of claim 19, wherein said second nanoparticle has substantially the same composition as said first nanoparticle.

21. The method of any one of claims 1-18, where said DNA or said DNA vector and said type 1 interferon receptor pathway inhibitor are provided together in said first nanoparticle.

22. The method of any one of claims 1-21, wherein said first nanoparticle is a lipid nanoparticle or a lipid polymer nanoparticle.

23. The method of claim 21 or 22, wherein said first nanoparticle is configured to release said type 1 interferon receptor pathway inhibitor prior to the release of said DNA or said DNA vector.

24. The method of any one of claims 1-23, wherein said type 1 interferon receptor pathway inhibitor is administered at about the same time up to about 4 hours prior to administration of said DNA or said DNA vector.

25. The method of any one of claims 1-24, wherein said DNA substantially comprises double-stranded DNA and said DNA vector substantially comprises double-stranded DNA.

26. The method of any one of claims 2-25, wherein said subject is a human patient, and said method provides a therapeutically effective amount of said transgene.

27. A nanoparticle composition comprising
a. a DNA; and
b. a type 1 interferon receptor pathway inhibitor.

28. The composition of claim 27, wherein said DNA is a DNA vector comprising a transgene operatively linked to a regulatory element.

29. The composition of claim 28, wherein said DNA vector comprises 5′ to 3′ a promoter, said transgene, and a polyadenylation signal sequence.

30. The composition of any one of claims 27-29, wherein said type 1 interferon receptor pathway inhibitor is a Janus activated kinase 1 inhibitor or a tyrosine kinase 2 inhibitor.

31. The composition of claim 30, wherein said type 1 interferon receptor pathway inhibitor is a compound of Table 1 or a pharmaceutically acceptable salt thereof.

32. The composition of any one of claims 27-29, wherein said type 1 interferon receptor pathway inhibitor is a signal transducer and activator of transcription protein (STAT) inhibitor.

33. The composition of claim 32, wherein said STAT inhibitor is a STATI or STAT2 inhibitor.

34. The composition of claim 33, wherein said STAT inhibitor is a compound of Table 2 or a pharmaceutically acceptable salt thereof.

35. The composition of any one of claims 27-34, wherein said transgene encodes a viral antigen, a bacterial antigen, a therapeutic protein, a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), or a transcription activator-like effector nuclease (TALEN).

36. The composition of any one of claims 27-35, wherein said nanoparticle is a lipid nanoparticle or a lipid polymer nanoparticle.

37. The composition of claim 36, wherein said nanoparticle is a lipid polymer nanoparticle configured to release said type 1 interferon receptor pathway inhibitor prior to said DNA vector.

38. The composition of any one of claims 27-37, wherein said DNA substantially comprises double-stranded DNA and said DNA vector substantially comprises double-stranded DNA.

39. A pharmaceutical composition comprising the nanoparticle composition of any one of claims 27-38 and a pharmaceutically acceptable carrier.

40. The pharmaceutical composition of claim 39, wherein said composition is for use in medicine or gene therapy.

41. A pharmaceutical composition for use in medicine, preferably gene therapy, comprising the first nanoparticle and DNA vector of any one of claims 2-26 for use with type 1 interferon receptor pathway inhibitor of any of claims 2-12.

42. A compound having the structure of: 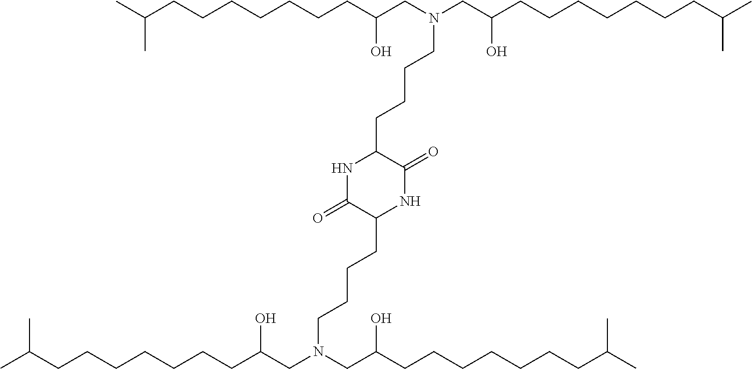 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]
or a pharmaceutically acceptable salt thereof.

43. The method of any one of claims 1-26 or composition of any one of claims 27-41, wherein said nanoparticle comprises the compound of claim 42.


SEQUENCE LISTING (2 sequences)

SEQ ID NO: 1 - DNA, 24 bp, synthetic construct
  source 1..24: mol_type = other DNA
  modified_base 1..24: mod_base = OTHER; note = phosphorothioate internucleotide linkage
ttagggttagggttagggttaggg

SEQ ID NO: 2 - DNA, 24 bp, synthetic construct
  source 1..24: mol_type = other DNA
ttagggttagggttagggttaggg